(19) United States
(12) Patent Application Publication
(10) Pub. No.: US 2025/0044289 A1
(43) Pub. Date: Feb. 6, 2025

(54) MOLECULARLY IMPRINTED POLYMER COATINGS AND SENSORS FOR BIODETECTION

(71) Applicant: ORCA HOLDINGS, LLC, Ketchikan, AK (US)

(72) Inventors: Pouya REZAI, Baltimore, MD (US); Satinder BRAR, Baltimore, MD (US); Ali DOOSTMOHAMMADI, Baltimore, MD (US); Shiva AKHTARIAN, Baltimore, MD (US); Khaled YOUSSEF, Baltimore, MD (US); Garrett KRAFT, Baltimore, MD (US)

(21) Appl. No.: 18/696,321

(22) PCT Filed: Sep. 28, 2022
(86) PCT No.: PCT/US2022/077198

§ 371 Date: Mar. 27, 2024

Related U.S. Application Data
(60) Provisional application No. 63/249,369, filed on Sep. 28, 2021.

(51) Int. Cl. G01N 33/569; G01N 33/543

(52) U.S. Cl. CPC G01N 33/56911; G01N 33/54373; G01N 33/56916; G01N 2333/195; G01N 2333/245; G01N 2600/00

(57) ABSTRACT

Provided herein are molecularly imprinted polymer (MIP) coated articles, sensors comprising polymer (MIP) coated articles, methods of manufacture and uses thereof.

### CROSS-REFERENCE TO RELATED APPLICATIONS

This application claims the benefit of and priority to U.S. Application Ser. No. 63/249,369, filed Sep. 28, 2021, the contents of which are hereby incorporated by reference in its entirety for all purposes.

### BACKGROUND

Infectious diseases caused by pathogens, including viruses, bacteria, and fungi have led to the loss of millions of lives. Coronavirus disease 2019 (COVID-19), the highly contagious viral illness caused by severe acute respiratory syndrome coronavirus 2 (SARS-CoV-2), has had a catastrophic effect on the world's demographics resulting in more than 3.8 million deaths worldwide, emerging as the most consequential global health crisis since the era of the influenza pandemic of 1918. Furthermore, several pathogenic bacteria strains, such as methicillin-resistant Staphylococcus aureus (MRSA) and Escherichia coli (O157:H7), have been reported to be present in soil, marine, and food which are the cause for serious life-threatening infectious diseases.

The ability to rapidly and selectively detect and identify pathogenic microorganisms and other biological macromolecules in clinical samples, food and the environment is of paramount importance in clinical diagnostics, environmental testing, and food security settings.

Conventionally, detection is achieved using plating\culturing, immunoassays, and biochemical tests. However, these techniques lack sensitivity and are expensive, laborious, time-consuming, and non-specific. Natural receptor-ligand interactions, such as antibodies and enzymes, have been extensively employed for affinity-based immunocapturing, but they suffer from limitations such as unstable over long periods and outside the physiological environment, and non-specific because of the surface affinity and selectivity of antibodies or enzymes.

There is a need for methods and materials for rapidly and selectively detecting biological targets including pathogenic microorganisms, such as bacteria and viruses.

### SUMMARY

In an aspect, the present disclosure provides a molecularly imprinted polymer (MIP) coated article comprising:
(a) a substrate; and
(b) a MIP coating on the substrate;
wherein the MIP coating is prepared by polymerizing a reaction mixture comprising one or more binding monomers, one or more crosslinking monomers, at least one initiator, and at least one template having substantially the same size and shape as at least one target, on the surface of the substrate;
wherein the MIP coating has a plurality of imprinted cavities which selectively bind the at least one target and one or more properties selected from the group consisting of:
(i) an average thickness of about 0.01 μm to 100 μm; and
(ii) a uniformity of 90% or more.

In some embodiments, the present disclosure provides a molecularly imprinted polymer (MIP) coated article comprising a substrate coated with a MIP polymer, prepared by a process comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as at least one target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
(i) an average thickness of 0.01 μm to 10 μm; and
(ii) a uniformity of 90% or more;
then removing the template from the polymer thereby forming a plurality of imprinted cavities in the MIP polymer which selectively bind at least one target.

In some embodiments, the present disclosure provides a molecularly imprinted polymer (MIP) coated article comprising a substrate coated with a MIP polymer, prepared by a process comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as at least one target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
(i) an average thickness of 0.01 m to 10 μm; and
(ii) a uniformity of 90% or more;

then removing the template from the polymer thereby forming a plurality of imprinted cavities in the MIP polymer which selectively bind at least one target.

In embodiments, the substrate has a curved surface, such as a microparticle or a microwire.

In embodiments, the target is a pathogen, such as a bacterium or a virus.

In embodiments, provided herein is a sensor comprising a MIP coated article of the present disclosure and a transduction device, wherein when a target selectively binds to at least a portion of the imprinted cavities of the MIP coated article, the transduction device produced a signal.

In embodiments, the present disclosure provides process for preparing a MIP coated article, comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as the target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 μm; and
   (ii) a uniformity of 90% or more;

then removing the at least one template from the MIP polymer layer thereby forming a plurality of imprinted cavities in the MIP polymer layer which selectively bind at least one target.

In embodiments, the present disclosure provides process for preparing a MIP coated article, comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as the target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 μm; and
   (ii) a uniformity of 90% or more;

then removing the at least one template from the MIP polymer layer thereby forming a plurality of imprinted cavities in the MIP polymer layer which selectively bind at least one target.

In embodiments, provided herein are methods for detecting a pathogen, comprising contacting a sensor of the present disclosure wherein the at least one target is a pathogen, with a fluid sample, wherein if the sample contains an amount of pathogen corresponding to at least the lower detection limit of the pathogen, the transduction device provides a signal indicating the presence of the pathogen, and if the sample contains an amount of pathogen below the lower detection limit of the pathogen, the transduction device does not provide a signal.

### BRIEF DESCRIPTION OF THE DRAWINGS

FIG. 1 is a schematic illustration of the preparation of molecularly imprinted core-shell microparticles described in Example 1.

FIG. 2 shows optical microscopy images of MIP-coated microparticles. A graph representing particle diameter for samples is shown. (0) shows uncoated CPS and (1) to (6) show coated particles using two-step temperature-rising polymerization method according to the samples in table 1 of Example 1.

FIG. 3 shows fluorescent images of the particles before (A) and after (B) MIP coating from Example 1.

FIG. 4. (A) and (B) depict imprinted core-shell particles, and imprinted polymer shell before template removal. (C) depicts a NIP coated particle, and (D) NIP core-shell microspheres (PS-NIP).

FIG. 5. shows (A) a fluorescent image of MIP coated particles after OP50 template removal, (B) shows SEM images of the MIP coated particles surface after OP50 template removal and (C) shows a complementary cavity on the MIP shell after OP50 template removal.

FIG. 6 shows E. coli OP50 concentration before and after bacteria capturing experiments using different concentrations of MIPs and NIPs. Uptake ratio was calculated as (n₀−n)/n₀ from the number of cells in the initial suspension (no) and in the supernatant after incubation with the microspheres (n).

FIG. 7 shows a time-lapse graph showing fluorescence changes of (A) bare particles in LB broth solution (zero concentration of bacteria), (B) bare particles exposed with 10⁵ (cells/mL) concentration of bacteria, (C) MIP-MPs exposed with 10⁵ (cells/mL) concentration of bacteria.

FIG. 8 shows MIP-MPs capturing performance in different concentration of bacteria. Error bars are standard error of the mean (SEM) and **: p<0.01, ***: p<0.001, ns: not significant.

FIG. 9 shows the uptake ratio for Sarcina imprinted microspheres at three different concentrations of 10²/mL, 10³/mL and 10⁴/mL compared to non-imprinted controls and E. coli imprinted microspheres in the binding test described in Example 4.

FIG. 10 shows % virus recovery rate of T4 phage cells (10³ PFU/mL) using the imprinted microspheres in three different concentrations of 10³/mL, 10⁴/mL and 10⁵/mL compared to non-imprinted controls in the dose-dependent binding test described in Example 5.

FIG. 11 shows % virus recovery of T4 phage cells (10³ PFU/mL) using the imprinted microspheres (10⁴/mL) compared to non-imprinted controls (10⁴/mL) at time points of 15 min, 30 min, 45 min, 1 h, 2 h, 3 h, 6 h, 9 h in the time-dependent binding test described in Example 5.

FIG. 12 shows % virus recovery T4 phage cells (10³ PFU/mL) using the imprinted microspheres (10⁴/mL) compared to non-imprinted controls at time points of 15 min, 30 min, 45 min, 1 h, 2 h, 3 h, 6 h, 9 h in the time-dependent binding test described in Example 5.

FIG. 13 shows the mean grey values (MGV, A.U.) of the coated microspheres, imprinted and washed MIP, and T4-bound MIP described in Example 5.

FIG. 14 is a schematic illustration of the preparation of molecularly imprinted polymers on microwires described in Example 6.

FIG. 15 shows rebinding assay using 10⁴ PFU/mL of E. coli OP50 described in Example 7.

FIG. 16 shows resistance measurements of microfluidic sensor exposed to 10⁴ PFU/mL of E. coli OP50 described in Example 7.

FIG. 17 shows fluorescent images of the particle surface of MIP coated SS wires with recipes from Example 6, Table 3.

FIG. 18 shows fluorescent images of the particle surface of MIP coated SS wires formed with MIPs made from different combinations of 2 functional monomer (A and D), 3 functional monomer (B and E), and 4-functional monomer systems (C and F).

FIG. 19 shows uptake ratio of Sarcina bacteria in Sarcina-templated stainless steel microwires.

FIG. 20 shows SEM images of bare, uncoated particles used in Example 2.

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, “sample” includes any sample containing or potentially containing a target. Samples contemplated herein include, but are not limited to, aerosols, fluid samples, solid samples, biological samples, food matrices, insects, and environmental samples (e.g., air, water, wastewater) containing or potentially containing a target etc. In some embodiments, the sample is droplets or fomites generated by, exhaled breath, coughing and/or sneezing which contain or potentially contain a target (e.g., target virus). In some embodiments, the sample is from environmental surfaces such as hospitals, schools/education environments, airports and airlines, medical devices, floor sweepings, bed railings, tables, bedding, cloths, door knobs, other industry environments such as cruise ships, mass transit, mass events like sporting events, hotels, food processing, critical supply chains, nursing homes, home testing, and the like. In some embodiments, the sample is from a plant which contains or potentially contains a target, e.g., a sample from a seed, stem, leaf, mushroom, and the like.

Biological samples, contemplated herein, include, for example, one or more biological samples selected from the group consisting of bioaerosols, biological fluids, tissue extracts and tissues. In embodiments, the bioaerosol is droplets or fomites generated by, e.g., exhaled breath, coughing and/or sneezing. In embodiments, the biological fluid can be selected from the group consisting of blood, cerebrospinal fluid, serum, plasma, urine, nipple aspirate, fine needle aspirate, tissue lavage, saliva, sputum, ascites fluid, semen, lymph node sample, vaginal pool, synovial fluid, spinal fluid, amniotic fluid, breast milk, pulmonary sputum or surfactant, urine, fecal matter, fluids collected from any of liver, kidney, breast, bone, bone marrow, testes, brain, ovary, skin, lung, prostate, thyroid, pancreas, cervix, stomach, intestine, colorectal, bladder, colon, uterus, head and neck, nasopharynx tumors, and other liquid samples of biologic origin.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

The ability to rapidly and selectively detect and identify pathogenic microorganisms and other biological macromolecules in clinical samples, food and the environment is of paramount importance in clinical diagnostics, environmental testing, and food security settings.

Conventionally, detection is achieved using plating\culturing, immunoassays, and biochemical tests. However, these techniques lack sensitivity and are expensive, laborious, time-consuming, and relatively non-specific. Biosensors rely on a natural biological molecular element and a transducer that changes its signal based on the interaction between the targeted molecule and the natural receptor. Natural receptor-ligand interactions, such as antibodies and enzymes, have been extensively employed for affinity-based immunocapturing, however, they are unstable over long periods and outside the physiological environment, and non-specific because of the surface affinity and selectivity of antibodies or enzymes.

Molecularly imprinted polymers are highly selective absorbents with absorption sites specifically tailored to bind to a particular target molecule. Examples of known MIPs and methods of preparing and using MIPs include those disclosed in U.S. Pat. Nos. 7,067,702; 7,319,038; 7,476,316; 7,678,870; 8,058,208; 8,591,842, 9,504,988, 6,582,971, 8,138,289, 6,127,154, 6,582,971, 6,884,842, and 7,285,219 and those disclosed in US publication Nos. 2010/0291224, 2016/0199752, 2010/0297610, 2017/0253647, 2009/0325147, and 2008/0033073 which are incorporated by reference herein in their entirety for all purposes.

Compared to the conventional immunoassays, MIPS possess various advantages of cost-effectiveness, ease of fabrication, enhanced thermal and chemical stability, reusability, and long shelf-time.

Conventionally, MIPs are synthesized through the bulk polymerization technique, which involves a combination of the template molecules, functional monomers, initiators and cross-linking reagents with a porogen solvent. The optimal form of MIPs is the spherical morphology, which is quite like antibodies, to accelerate binding kinetics and binding-site accessibility, and to ease the deposition on the exterior of nano-devices. Therefore, the obtained monolithic layer through bulk polymerization is grinded, crushed, and filtered to obtain an appropriate spherical morphology. However, this process suffers from shortcomings, such as irregular particle shapes with low reproducibility, low binding capacity, rough elution of the template molecules, incomplete template removal, poor site accessibility and large mass transfer resistance, to name a few. Other procedures like emulsion polymerization, suspension polymerization and precipitation polymerization have been employed however, the obtained microspheres were polydisperse in size and contained residual surfactants in their surfaces that can reduce selectivity of the target species, resulting in low binding and recognition capacity. Furthermore, it is difficult to adjust and control the final particle size without deteriorating the imprinting effect.

Non-planar substrates such as core-shell MIPs and cylindrical microwires have the potential to offer advantages over flat-surface MIP substrates for the identification and quantification of biological molecules and cells including for example, smaller amount of samples used, and the possibility of suspending non-planar MIP substrates in samples to enhance capturing opportunities and achieve better detection limits. Obtaining a MIP polymer layer of a specific thickness based on template size would be beneficial for tailor-forming the recognition sites according to the size and shape of the molecular template, and also beneficial for target detection (e.g., by fluorescence) to maximize the intensity difference before and after target capture.

Accordingly, it would be desirable to prepare MIP absorbents which can be coated on substrates, such as non-planar substrates with controllable chemical properties, material properties and physical mechanical properties, and display high selectivity and sensitivity for the target, and the integration of such materials in portable biosensors for the rapid detection of biological targets. The methods and materials of the present disclosure provide such improvements over conventional MIP materials and processes.

### Molecularly Imprinted Polymer Coatings & Methods of Manufacture

The materials and methods disclosed herein are useful in various applications not only in molecular imprinting methods, but also in the coating of various functional layers, such as magnetic and fluorescent layers, on colloidal particles or planar substrates.

In embodiments, the present disclosure provides a molecularly imprinted polymer (MIP) coated article comprising:
(a) a substrate; and
(b) a MIP coating on the substrate;
wherein the MIP coating is prepared by polymerizing a reaction mixture comprising one or more binding monomers, one or more crosslinking monomers, at least one initiator, and at least one template having substantially the same size and shape as at least one target, on the surface of the substrate;
   wherein the MIP coating has a plurality of imprinted cavities which selectively bind the at least one target and one or more properties selected from the group consisting of:
      (i) an average thickness of 0.01 μm to 10 μm;
      (ii) a uniformity of 90% or more;
      iii) a porosity of 1 m²/g-1000 m²/g;
      (iv) a cavity size of 10 nm to 500 μm; and
      (v) a crosslinking density of 10%-90%.

In embodiments, the average thickness is about 5 μm. In embodiments, the average thickness is about 1 μm to about 10 μm.

In embodiments, the present disclosure provides a molecularly imprinted polymer (MIP) coated article comprising:
(a) a substrate; and
(b) a MIP coating on the substrate;
wherein the MIP coating is prepared by polymerizing a reaction mixture comprising one or more binding monomers, one or more crosslinking monomers, at least one initiator, and at least one template having substantially the same size and shape as at least one target, on the surface of the substrate;
   wherein the MIP coating has a plurality of imprinted cavities which selectively bind the at least one target and one or more properties selected from the group consisting of:
      (i) an average thickness of 0.01 μm to 10 μm;
      (ii) a uniformity of 90% or more;
      iii) a porosity of 1 m²/g-1000 m²/g;
      (iv) a cavity size of 10 nm to 500 μm; and
      (v) a crosslinking density of 10%-90%.

In embodiments, the present disclosure provides a molecularly imprinted polymer (MIP) coated article comprising:
(a) a substrate; and
(b) a MIP coating on the substrate;
wherein the MIP coating is prepared by polymerizing a reaction mixture comprising one or more binding monomers, one or more crosslinking monomers, at least one initiator, and at least one template having substantially the same size and shape as at least one target, on the surface of the substrate;
   wherein the MIP coating has a plurality of imprinted cavities which selectively bind the at least one target and one or more properties selected from the group consisting of:
      (i) an average thickness of 0.01 μm to 10 μm; and
      (ii) a uniformity of 90% or more.

In embodiments, the present disclosure provides a molecularly imprinted polymer (MIP) coated article comprising:
(a) a substrate; and
(b) a MIP coating on the substrate;
wherein the MIP coating is prepared by polymerizing a reaction mixture comprising one or more binding monomers, one or more crosslinking monomers, at least one initiator, and at least one template having substantially the same size and shape as at least one target, on the surface of the substrate;
   wherein the MIP coating has a plurality of imprinted cavities which selectively bind the at least one target and one or more properties selected from the group consisting of
      (i) an average thickness of 0.01 μm to 10 μm; and
      (ii) a uniformity of 90% or more.

In embodiments, the average thickness is about 5 μm. In embodiments, the average thickness is about 1 μm to about 10 μm.

In embodiments, provided herein is a molecularly imprinted polymer (MIP) coated article comprising a substrate coated with a MIP polymer, prepared by a process comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as at least one target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 μm;
   (ii) a uniformity of 90% or more;
   (iii) a porosity of 1 m²/g-1000 m²/g;
   (iv) a cavity size of 10 nm to 500 μm; and
   (v) a crosslinking density of 10%-90%;
then removing the template from the polymer thereby forming a plurality of imprinted cavities in the MIP polymer which selectively bind at least one target.

In embodiments, provided herein is a molecularly imprinted polymer (MIP) coated article comprising a substrate coated with a MIP polymer, prepared by a process comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as at least one target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 μm; and
   (ii) a uniformity of 90% or more;
then removing the template from the polymer thereby forming a plurality of imprinted cavities in the MIP polymer which selectively bind at least one target.

In embodiments, the average thickness is about 5 μm. In embodiments, the average thickness is about 1 μm to about 10 μm.

In embodiments, provided herein is a molecularly imprinted polymer (MIP) coated article comprising a substrate coated with a MIP polymer, prepared by a process comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as at least one target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 am; and
   (ii) a uniformity of 90% or more;
then removing the template from the polymer thereby forming a plurality of imprinted cavities in the MIP polymer which selectively bind at least one target.

In embodiments, the MIP coated polymer layer is formed in one polymerization step.

In embodiments, the first polymerization step (c) is carried out at a temperature between 20° C. to about 70° C., including from about 20° C., about 25° C., about 30° C., about 35° C., about 40° C., about 45° C., about 50° C., about 55° C., about 60° C., about 65° C., about 70° C., including all values and subranges therebetween.

In embodiments, the first polymerization step (c) is carried out for about or at least about 5 min to about or at least about 12 h or more, including from about or at least about 5 min, about or at least about 10 min, about or at least about 30 min, about or at least about 45 min, about or at least about 1 hour, about or at least about 2 hour, about or at least about 3 hour, about or at least about 4 hours, about or at least about 5 hours, about or at least about 6 hours, about or at least about 7 hours, about or at least about 8 hours, about or at least about 9 hours, about or at least about 10 hours, about or at least about 11 hours, to about or at least 12 hours or more, including all values and ranges therebetween.

In embodiments, the second polymerization step (d) is carried out at a temperature is between 30° C. to about 100° C., including from about 30° C., about 35° C., about 40° C., about 45° C., about 50° C., about 55° C., about 60° C., about 65° C., about 70° C., about 75° C., about 80° C., about 85° C., about 90° C., about 95° C., to about 100° C., including all values and subranges therebetween.

In embodiments, the second polymerization step (c) is carried out for about or at least about 5 min to about or at least about 24 h or more, including from about or at least about 5 min, about or at least about 10 min, about or at least about 30 min, about or at least about 45 min, about or at least about 1 hour, about or at least about 2 hour, about or at least about 3 hour, about or at least about 4 hours, about or at least about 5 hours, about or at least about 6 hours, about or at least about 7 hours, about or at least about 8 hours, about or at least about 9 hours, about or at least about 10 hours, about or at least about 11 hours, to about or at least 12 hours, about or at least about 14 hours, about or at least about 16 hours, about or at least about 18 hours, about or at least about 20 hours, about or at least about 22 hours, about or at least about 24 hours or more, including all values and ranges therebetween.

In embodiments of the MIP coated article described herein, one or more of steps (c) and (d) are carried out under the following conditions:
(i) said first polymerization conditions of step (c) is carried out at a temperature of about 20° C. to about 50° C.; and
(ii) said second polymerization conditions of step (d) is carried out at a temperature of about 50° C. to about 70° C.

In embodiments of the MIP coated articles described herein, one or more of steps (c) and (d) are carried out under the following conditions:
(i) said first polymerization conditions of step (c) is carried out at a temperature of about 40° C. to about 50° C.; and
(ii) said second polymerization conditions of step (d) is carried out at a temperature of about 50° C. to about 60° C.

In embodiments of the MIP coated articles described herein, one or more of steps (c) and (d) are carried out under the following conditions:
(i) said first polymerization conditions of step (c) is carried out at a temperature of about 40° C.; and
(ii) said second polymerization conditions of step (d) is carried out at a temperature of about 50° C.

In embodiments of the MIP coated articles described herein, one or more of steps (c) and (d) are carried out under the following conditions:
(i) said first polymerization conditions of step (c) is carried out at a temperature of about 50° C.; and
(ii) said second polymerization conditions of step (d) is carried out at a temperature of about 60° C.

In embodiments of the MIP coated articles described herein, one or more of steps (c) and (d) are carried out under the following conditions:
(i) said first polymerization conditions of step (c) is carried out at a temperature of about 40° C. to about 50° C. for about 4 to 6 hours; and
(ii) said second polymerization conditions of step (d) is carried out at a temperature of about 50° C. to about 60° C. for at least about 2 to 10 hours.

In embodiments of the MIP coated articles described herein, steps (c) and (d) are carried out under the following conditions:
(i) said first polymerization conditions of step (c) is carried out at a temperature of about 40° C.; for about 6 hours; and
(ii) said second polymerization conditions of step (d) is carried out at a temperature of about 50° C. for about 6 hours.

In embodiments of the MIP coated articles described herein, steps (c) and (d) are carried out under the following conditions:
(i) said first polymerization conditions of step (c) is carried out at a temperature of about 40° C.; for about 6 hours; and
(ii) said second polymerization conditions of step (d) is carried out at a temperature of about 50° C. for about 8 hours.

In embodiments of the MIP coated articles described herein, steps (c) and (d) are carried out under the following conditions:
(i) said first polymerization conditions of step (c) is carried out at a temperature of about 40° C.; for about 6 hours; and
(ii) said second polymerization conditions of step (d) is carried out at a temperature of about 50° C. for about 10 hours.

In embodiments of the MIP coated articles described herein, steps (c) and (d) are carried out under the following conditions:
(i) said first polymerization conditions of step (c) is carried out at a temperature of about 50° C.; for about 4 hours; and
(ii) said second polymerization conditions of step (d) is carried out at a temperature of about 60° C. for about 2 hours.

In embodiments of the MIP coated articles described herein, steps (c) and (d) are carried out under the following conditions:
(i) said first polymerization conditions of step (c) is carried out at a temperature of about 50° C.; for about 4 hours; and
(ii) said second polymerization conditions of step (d) is carried out at a temperature of about 60° C. for about 3 hours.

In embodiments of the MIP coated articles described herein, steps (c) and (d) are carried out under the following conditions:
(i) said first polymerization conditions of step (c) is carried out at a temperature of about 50° C.; for about 4 hours; and
(ii) said second polymerization conditions of step (d) is carried out at a temperature of about 60° C. for about 4 hours.

In embodiments of the MIP coated articles described herein, when (i) said first polymerization conditions of step (c) is carried out at a temperature of about 40° C.; for about 6 hours; and (ii) said second polymerization conditions of step (d) is carried out at a temperature of about 50° C. for about 6 hours, a MIP polymer layer having an average thickness of about 0.5 μm±0.5 μm is formed.

In embodiments of the MIP coated articles described herein, when (i) said first polymerization conditions of step (c) is carried out at a temperature of about 40° C.; for about 6 hours; and (ii) said second polymerization conditions of step (d) is carried out at a temperature of about 50° C. for about 8 hours, a MIP polymer layer having an average thickness of about 1.95 μm±0.5 μm is formed.

In embodiments of the MIP coated articles described herein, when (i) said first polymerization conditions of step (c) is carried out at a temperature of about 40° C.; for about 6 hours; and (ii) said second polymerization conditions of step (d) is carried out at a temperature of about 50° C. for about 10 hours, a MIP polymer layer having an average thickness of about 2.65 μm±0.5 μm is formed.

In embodiments of the MIP coated articles described herein, when (i) said first polymerization conditions of step (c) is carried out at a temperature of about 50° C.; for about 4 hours; and (ii) said second polymerization conditions of step (d) is carried out at a temperature of about 60° C. for about 2 hours, a MIP polymer layer having an average thickness of about 3.5 μm±0.5 μm is formed.

In embodiments of the MIP coated articles described herein, when (i) said first polymerization conditions of step (c) is carried out at a temperature of about 50° C.; for about 4 hours; and (ii) said second polymerization conditions of step (d) is carried out at a temperature of about 60° C. for about 3 hours, a MIP polymer layer having an average thickness of about 4.9 μm±0.6 μm is formed.

In embodiments of the MIP coated articles described herein, when (i) said first polymerization conditions of step (c) is carried out at a temperature of about 50° C.; for about 4 hours; and (ii) said second polymerization conditions of step (d) is carried out at a temperature of about 60° C. for about 4 hours, a MIP polymer layer having an average thickness of about 6 μm±1.1 μm is formed.

In embodiments, any solvent which provides suitable solubility and is compatible with the desired reaction to the conditions to form the MIP materials of the present disclosure may be used. For example, the solvent can include polar protic solvent or a polar aprotic solvent. In embodiments, the solvent is an alcohol solvent, such as methanol, ethanol, or isopropanol. In embodiments, the solvent is DMSO. In embodiments, the solvent is acetonitrile.

In embodiments of the MIP coated article described herein, one or more of steps (b), (c), and (d) is carried out in a porogen solvent. In embodiments, steps (b), (c), and (d) are carried out in a porogen solvent. In embodiments the porogen solvent is DMSO. In embodiments, the porogen solvent is acetonitrile. In embodiments, the porogen solvent is an alcohol. In embodiments, the porogen solvent is toluene. In embodiments, the porogen solvent is PBS.

In embodiments, the molecularly imprinted polymers are in the form of beads, particularly porous beads that have sufficient porosity so as to allow facile mass transport in and out of the bead.

In embodiments, the molecularly imprinted polymers of the present disclosure are in the form of a thin film. In some embodiments, the molecularly imprinted polymers of the present disclosure is in the form of a coating.

In embodiments, the molecularly imprinted polymers of the present disclosure are in the form of molecularly imprinted nano-particles (e.g. imprinted core shell microspheres).

In embodiments, the present disclosure provides a process for preparing a MIP coated article, comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as the target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 μm;
   (ii) a uniformity of 90% or more;
   iii) a porosity of 1 m²/g-1000 m²/g;
   (iv) a cavity size of 10 nm to 500 μm; and
   (v) a crosslinking density of 10%-90%.
then removing the at least one template from the MIP polymer layer thereby forming a plurality of imprinted cavities in the MIP polymer layer which selectively bind at least one target.

In embodiments, the present disclosure provides a process for preparing a MIP coated article, comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as the target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 μm;
   (ii) a uniformity of 90% or more;
   iii) a porosity of 1 m²/g-1000 m²/g;
   (iv) a cavity size of 10 nm to 500 μm; and
   (v) a crosslinking density of 10%-90%.
then removing the at least one template from the MIP polymer layer thereby forming a plurality of imprinted cavities in the MIP polymer layer which selectively bind at least one target.

In embodiments, the average thickness is about 5 μm. In embodiments, the average thickness is about 1 μm to about 10 μm.

In embodiments, the present disclosure provides a process for preparing a MIP coated article, comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as the target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 μm; and
   (ii) a uniformity of 90% or more;
then removing the at least one template from the MIP polymer layer thereby forming a plurality of imprinted cavities in the MIP polymer layer which selectively bind at least one target.

In embodiments, the average thickness is about 5 m. In embodiments, the average thickness is about 1 μm to about 10 μm.

In embodiments, the present disclosure provides a process for preparing a MIP coated article, comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as the target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
   (i) an average thickness of 0.01 μm to 10 μm; and
   (ii) a uniformity of 90% or more;
then removing the at least one template from the MIP polymer layer thereby forming a plurality of imprinted cavities in the MIP polymer layer which selectively bind at least one target.

In embodiments, MIP coated articles can be made from a starting substrate e.g., solid substrate and the MIP can be coated, adsorbed, or chemically attached to the surface of the substrate. In embodiments, the substrate is non-planar. In embodiments the substrate has a curved surface e.g., is spherical or cylindrical. In embodiments the substrate is a metal. In embodiments the substrate is non-metallic.

Suitable substrates contemplated for use herein include: glass, polystyrene spheres with fluorescent and/or magnetic cores, Fe₃O₄ nanoparticles, silica microspheres, polymeric beads, quantum dots, TiO₂ particles, Au nanoparticles, gold and silver nanoclusters, magnetic nanoparticles, polyamide microspheres and microcapsules, stainless steel wires, copper fibers, microwires, alumina, and quartz, or other inorganic supports.

In embodiments, the substrate is a microparticle or a microwire. In embodiments, the substrate is a spherical microparticle. In embodiments, the substrate is a cylindrical microwire. In embodiments, the substrate is a magnetic fluorescent polystyrene microparticle. In embodiments, the substrate is a carboxyl magnetic polystyrene bead.

In embodiments, the surface of the substrate is functionalized to react with the MIP coating. For example, the substrate may be modified with carboxyl groups, amine groups, hydroxyl groups, or silylate groups. In embodiments, the substrate is surface treated e.g., by oxidation or silylation using conditions known in the art. In embodiments, the substrate is surface functionalized with carboxyl groups.

In embodiments, the substrate is a stainless-steel wire surface treatment by oxidation and silylation.

In embodiments, the substrate is first modified with a polymer layer formed from a single monomer, and subsequently coated with a polymer formed from multi-monomer mixture (e.g., as described herein) on top of the layer formed from a single monomer.

The complexing cavity may have a geometry (including its size and shape) that is complementary to the template. The shape of the complexing cavity will vary depending on the template. For example, viruses are known to have various shapes, including spherical, icosahedral, or rod-like; and bacteria are known to have shapes including spherical (e.g., coccus, diplococci, tetrad, sarcina, staphylococci, and streptococci), rod-shaped (e.g., bacillus, diplobacillus, streptobacillus, palisade, coccobacillus, vibrio), and spiral shapes (e.g., spirillum, spirochete, helical), filamentous, box-shaped (Arcula), pleomorphic. As such, where the template is a virus, the shape of the complexing cavity can be spherical, icosahedral, or rod-like, and where the template is a bacteria the shape of the complexing cavity can be spherical (e.g., coccus, diplococci, tetrad, sarcina, staphylococci, and streptococci), rod-shaped (e.g., bacillus, diplobacillus, streptobacillus, palisade, coccobacillus, vibrio), and spiral shapes (e.g., spirillum, spirochete, helical), filamentous, box-shaped (Arcula), pleomorphic and the like.

The size of the complexing cavity will also vary depending upon the template. In some cases, the size of the complexing cavity (as measured along its longest axis) can range from 1 nm-100 μm, including all values and subranges therebetween. In some cases, the size of the complexing cavity (as measured along its longest axis) can range from 10 nm-500 nm; complexing cavities having a size in this range may be useful for selectively binding viruses, which generally also have a size in this range. In embodiments, the complexing cavity is between 0.2 μm to 50 μm, or 0.2-20 μm (as measured along its longest axis); complexing cavities having this range may be useful for binding bacteria, which generally have a size in this range. For example, the average diameter of spherical bacteria is typically in the range of 0.5-2.0 μm. For rod-shaped or filamentous bacteria, length is typically in the range of 1-10 μm and diameter is 0.25-1.0 μm. The complexing cavity may be contained in the polymer matrix at any of various locations, including within the polymer matrix (e.g., completely enclosed within the polymer matrix) or at a surface of the polymer matrix (e.g., partially enclosed by the polymer matrix).

In embodiments, the target itself is used as a template.

In embodiments, the target is E. coli and the template is E. coli. In embodiments, the target is a Sarcina spp. E.g., Sarcina lutea and the template is a Sarcina spp. E.g., Sarcina lutea.

In embodiments, a suitable “surrogate or “template” for the target is used as the template. For example, a MIP selective for target “A” can be prepared by polymerizing a complex of a suitable surrogate “B” with a mixture of monomers as described herein, provided that target “A” and surrogate “B” complex to the binding monomer with at least some of the same characteristics, so the complexing cavities of the MIP would interact with the surrogate in a manner similar to that of the target e.g., using the same physicochemical mechanism, similar charge, morphology, size and/or shape. In embodiments, the template has substantially the same size and shape as the target.

In embodiments, when the target is one or more bacteria, the template has substantially the same size and/or shape as the bacteria target. For example, the template can be spherical (e.g., coccus, diplococci, tetrad, sarcina, staphylococci, and streptococci), rod-shaped (e.g., bacillus, diplobacillus, streptobacillus, palisade, coccobacillus, vibrio), and spiral shapes (e.g., spirillum, spirochete, helical), filamentous, box-shaped (Arcula), pleomorphic and the like, and/or the size of the template (as measured along its longest axis) can range from 0.5 to 50 μm, including all values and subranges therebetween.

In embodiments, when the target is a bacteria the template is a non-pathogenic strain of the target bacteria.

In embodiments the target is E. coli and the template is non-pathogenic E. coli OP50 bacteria strain.

In embodiments, when the target is one or more viruses of a target viral genus, the template has substantially the same size and/or shape as the target virus(es). For example, the template can be spherical, icosahedral, or rod-like, and/or size of the template (as measured along its longest axis) can range from 10 nm-500 nm, including all values and subranges therebetween.

In embodiments, when the target is one or more viruses of a target viral genus, the template can be an attenuated strain of the target virus which retains, for example the shell shape of the virus.

In embodiments, the template is bacteriophage T4.

In embodiments, suitable templates include macromolecules associated with the target, e.g., a polysaccharide group of a glycoprotein macromolecule, or analog thereof associated with a target virus. In embodiments, the template is a protein or surface feature of the virus (such as the spike protein). In some embodiments, suitable surrogates include micelles with expressed viral proteins, such as assembled proteins of viral capsid. In some embodiments, the surrogate is an antibody or portion of an antibody of a target virus. In some embodiments, the surrogate is a surface modified dendrimer.

In embodiments, the size of the template is between 10%-200% of the size of the target, including between about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, to about 200% of the target, including all values and subranges therebetween.

As needed, the template can be removed from the MIP material before use or can remain in place prior to use using any suitable conditions known in the art for example, stripping the template with an acidic solution or using base hydrolysis. For example, bacterial templates can be removed by oxidizing the polymer in an acidic or basic solution e.g., using methanol and acetic acid. In embodiments when the target is for example, a virus, the stripping step may comprise contacting the molecularly imprinted polymer bound to the target virus with acidic solution (e.g., aqueous inorganic acid or organic acid, such as HCl, acetic acid) to denature the virus and washing with deionized water to remove/elute the virus. In some embodiments, the stripping step comprises a saltwater wash. Other conditions to remove the template from the imprinted polymer materials, include incubating the MIP material with 1M HCl and 0.01% Triton X-100 under rotating conditions (e.g., 60 rpm, RT), centrifuged and washed with PBS.

In general, the binding monomer(s) used herein will have physicochemical properties suitable for interacting with the target, such as functional groups that can act as hydrogen bond donors or receptors, functional groups that can participate in ionic interactions, hydrophilic or hydrophobic interactions (e.g., Van der Waals interactions), pi-stacking, polar interactions, non-polar interactions, and the like. In some embodiments, the binding monomer can include multiple functional groups capable of interacting with the target using two or more such interactions. Alternatively (or in combination), the MIP can be prepared from a mixture of different binding monomers, each capable of interacting with the target via different physicochemical interactions.

In embodiments, at least one or more, or at least two or more, or at least three or more, or at least four or more binding monomers e.g., as described herein are used for synthesizing MIPs of the present disclosure.

In embodiments, 1-10 binding monomers e.g., as described herein are used for synthesizing MIPs of the present disclosure, including 1, 2, 3, 4, 5, 6, 7, 8, 9, or 10 binding monomers. In embodiments, 1 binding monomer is used for synthesizing MIPs of the present disclosure. In embodiments, 2 binding monomers are used for synthesizing MIPs of the present disclosure. In embodiments, 3 binding monomers are used for synthesizing MIPs of the present disclosure. In embodiments, 4 binding monomers are used for synthesizing MIPs of the present disclosure.

In embodiments, the one or more binding monomers are each independently selected from the group consisting of: a charged monomer, an uncharged polar monomer and an uncharged hydrophobic monomer. In embodiments, the selection of binding monomers has functionality complementary to the target or a portion of the target.

In embodiments, the charged binding monomer comprises an anionic functional group. For example, the monomer comprises a carboxylate, a sulfonate, or a phosphate.

In embodiments, the charged binding monomer comprises a cationic functional group, for example a quaternary ammonium ion, pyridinium, pyrollidinium, imidizolium, guanidinium, phosphonium or sulfonium.

A wide variety of monomers may be used as binding monomers for synthesizing the MIP materials in accordance with the present disclosure. Suitable non-limiting examples of binding monomers or non-crosslinking monomers that can be used for preparing a MIP of the present disclosure include one or more monomers independently selected from: methylmethacrylate, other alkyl methacrylates, alkylacrylates, allyl or aryl acrylates and methacrylates, cyanoacrylate, styrene, substituted styrenes, methyl styrene (multisubstituted) including 1-methylstyrene; 3-methylstyrene; 4-methylstyrene, etc.; vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy) ethyl methacrylate; 1-acetoxy-1,3-butadiene; 2-acetoxy-3-butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; aeryloyl chloride; 1-α-acryloyloxy-β,β-dimethyl-γ-butyrolactone; N-acryloxy succinimide acryloxytris(hydroxymethyl)amino-methane; N-acryloyl chloride; N-acryloyl pyrrolidinone; N-acryloyl-tris(hydroxymethyl)amino methane; 2-aminoethyl methacrylate; N-(3-aminopropyl)methacrylamide; (o, m, or p)-amino-styrene; t-amyl methacrylate; 2-(1-aziridinyl)ethyl methacrylate; 4-benzyloxy-3-methoxystyrene; 2-bromoacrylic acid; 4-bromo-1-butene; 3-bromo-3,3-difluoropropane; 6-bromo-1-hexene; 3-bromo-2-methacrylonitrile; 2-(bromomethyl)acrylic acid; 8-bromo-1-octene; 5-bromo-1-pentene; cis-1-bromo-1-propene; -bromostyrene; p-bromostyrene; bromotrifluoro ethylene; (±)-3-buten-2-ol; 1,3-butadiene; 1,3-butadiene-1,4-dicarboxylic acid 3-butenal diethyl acetal; 1-butene; 3-buten-2-ol; 3-butenyl chloroformate; 2-butylacrolein; t-butylacrylamide; butyl acrylate; butyl methacrylate; (o,m,p)-bromo styrene; t-butyl acrylate; 1-carvone; (S)-carvone; (−)-carvyl acetate; 3-chloroacrylic acid; 2-chloroacrylonitrile; 2-chloroethyl vinyl ether; 2-chloromethyl-3-trimethylsilyl-1-propene; 3-chloro-1-butene; 3-chloro-2-chloromethyl-1-propene; 3-chloro-2-methyl propene; 2,2-bis(4-chlorophenyl)-1,1-dichloroethylene; 3-chloro-1-phenyl-1-propene; m-chlorostyrene; o-chlorostyrene; p-chlorostyrene; 1-cyanovinyl acetate; 1-cyclopropyl-1-(trimethylsiloxy)ethylene; 2,3-dichloro-1-propene; 2,6-dichlorostyrene; 1,3-dichloropropene; 2,4-diethyl-2,6-heptadienal; 1,9-decadiene; 1-decene; 1,2-dibromoethylene; 1,1-dichloro-2,2-difluoroethylene; 1,1-dichloropropene; 2,6-difluorostyrene; dihydrocarveol; (±)-dihydrocarvone; (−)-dihydrocarvyl acetate; 3,3-dimethylacrylaldehyde; N,N′-dimethylacrylamide; 3,3-dimethylacrylic acid; 3,3-dimethylacryloyl chloride; 2,3-dimethyl-1-butene; 3,3-dimethyl-1-butene; 2-dimethyl aminoethyl methacrylate; 1-(3-butenyl)-4-vinylbenzene; 2,4-dimethyl-2,6-heptadien-1-ol; 2,4-dimethyl-2,6-heptadienal; 2,5-dimethyl-1,5-hexadiene; 2,4-dimethyl-1,3-pentadiene; 2,2-dimethyl-4-pentenal; 2,4-dimethylstyrene; 2,5-dimethylstyrene; 3,4-dimethylstryene; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethy 1-2-(hydroxymethyl)-1,3-propanediol trimethacrylate; ethyl methacrylate; ethyl vinyl ether; ethyl vinyl ketone; ethyl vinyl sulfone; (1-ethylvinyl)tributyl tin; m-fluorostyrene; o-fluorostyrene; p-fluorostyrene; glycol methacrylate (hydroxyethyl methacrylate); GA GMA; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; 1-hexadecene; 1,5-hexadien-3,4-diol; 1,4-hexadiene; 1,5-hexadien-3-ol; 1,3,5-hexatriene; 5-hexen-1,2-diol; 5-hexen-1-ol; hydroxypropyl acrylate; 3-hydroxy-3,7,11-trimethyl-1,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopulegol; itaconic acid; itaconalyl chloride; (±)-linalool; linalyl acetate; p-mentha-1,8-diene; p-mentha-6,8-dien-2-ol; methyleneamino acetonitrile; methacrolein; [3-(methacryloylamino)-propyl]trimethylammonium chloride; methacrylamide; methacrylic acid; methacrylic anhydride; methacrylonitrile; methacryloyl chloride; 2-(methacryloyloxy)ethyl acetoacetate; (3-meth-acryloxypropyl)trimethoxy silane; 2-(methacryloxy)ethyl trimethylammonium methylsulfate; 2-methoxy propene (isopropenyl methyl ether); methyl-2-(bromomethyl)acrylate; 5-methyl-5-hexen-2-one; methyl methacrylate; N,N′methylene bisacrylamide; 2-methylene glutaronitrite; 2-methylene-1,3-propanediol; 3-methyl-1,2-butadiene; 2-methyl-1-butene; 3-methyl-1-butene; 3-methyl-1-buten-1-ol; 2-methyl-1-buten-3-yne; 2-methyl-1,5-heptadiene; 2-methyl-1-heptene; 2-methyl-1-hexene; 3-methyl-1,3-pentadiene; 2-methyl-1,4-pentadiene; (±)-3-methyl-1-pentene; (±)-4-methyl-1-pentene; (±)-3-methyl-1-penten-3-ol; 2-methyl-1-pentene; methyl vinyl ether; methyl-2-vinyloxirane; methyl vinyl sulfone; 4-methyl-5-vinylthiazole; myrcene; t-nitrostyrene; 3-nitrostyrene; 1-nonadecene; 1,8-nonadiene; 1-octadecene; 1,7-octadiene; 7-27ctane-1,2-diol; 1-octene; 1-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-1-ol; 4-penten-2-ol; 4-phenyl-1-butene; phenyl vinyl sulfide; phenyl vinyl sulfonate; 2-propene-1-sulfonic acid sodium salt; phenyl vinyl sulfoxide; 1-phenyl-1-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyanoethylene; trans 3-chloroacrylic acid; 2-trifluoromethyl propene; 2-(trifluoromethyl)propenoic acid; 2,4,4′-trimethyl-1-pentene; 3,5-bis(trifluoromethyl)styrene; 2,3-bis(trimethylsiloxy)-1,3-butadiene; 1-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2-chloroethylsulfone; 4-(vinyl benzyl)-2-chloroethyl sulfone; N-(p-vinylbenzyl)-N,N′-dimethyl amine; 4-vinyl biphenyl (4-phenylstyrene); vinyl bromide; 2-vinyl butane; vinyl butyl ether; 9-vinyl carbazole; vinyl carbinol; vinyl cetyl ether; vinyl chloroacetate; vinyl hloroformate; vinyl crotanoate; vinyl peroxcyclohexane; 4-vinyl-1-cyclohexene; 4-vinylcyclohexene dioxide; vinyl cyclopentene; vinyl dimethylchlorosilane; vinyl dimethylethoxysilane; vinyl diphenylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinylquinoline; vinyl iodide; vinyllaurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbomene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinylquinoline; 1-vinyl silatrane; vinyl sulfone; vinyl sulfonic acid sodium salt; a-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy) silane; vinyl 2-valerate; vinyl benzoic acid; and vinyl imidazole, dopamine (DA); and the like.

In some embodiments, suitable binding monomers for synthesizing MIP materials in accordance with the present disclosure include acrylamide, methacrylic acid, methylmethacrylate, N-vinylpyrrolidone, acrylic acid, N-benzylacrylamide, Ethylene glycol dimethacrylate (EDMA); tetraethyl orthosilicate (TEOS); piperazine diacrylamide; 3-aminophenylboronic acid (APBA); 3-Aminopropyltriethoxysilane (APTES); polydopamine (PDA); N-methacryloyl-L-tyrosine methyl ester; hydroxyethylmethacrylate (HEMA); N-Isopropylacrylamide (NIPAM); acrylic acid (AA); polyurethane (PU); bisphenol A; phloroglucinol; N-tert butyl acrylamide; poly(tert-butylmethacrylate)-block-poly(2-hydroxyethyl methacrylate); allylamine; 1-vinyl imidazole; aminoethylacrylamide; (3-acrylamidopropyl) trimethyl ammonium chloride; N-(3-aminopropyl)methacrylamide; N-tert-butyl acrylamide; N-phenylacrylamide; N-hydroxymethylacrylamide; epichlorohydrin; sulphonic acids (e.g., 2-acrylamido-2-methylpropane sulphonic acid); carboxylic acids (e.g., acrylic acid, vinylbenzoic acid); heteroaromatic bases (e.g., vinylpyridine, vinylimidazole); polyvinylpyrrolidone (PVP), dimethylamino; ethyl methacrylate (DMAEMA), and polyamine (PA).

In embodiments, the charged binding monomer is acrylic acid or an alkylacrylic acid. In embodiments, the charged binding monomer is methacrylic acid.

In embodiments, the one or more hydrophilic binding monomers and one or more hydrophobic binding monomers are selected from the group consisting of acrylamide, methacrylamide, vinylpyridine, N-vinylpyrrolidone, N-alkylacrylamides, hydroxyethylmethacrylate, alkylineihacrylates, ethlmethacrylate and combinations thereof.

In embodiments, the MIP coating is prepared by polymerizing on the surface of the substrate a mixture comprising:
(i) one or monomers selected from the group consisting of acrylamide, methacrylic acid, methylmethacrylate, and N-vinylpyrrolidone, and combinations thereof,
(ii) one or more crosslinking monomers,
(iii) at least one initiator, and
(iv) at least one template having substantially the same size and shape as the target.

In some embodiments, a MIP of the present disclosure is prepared by polymerizing a mixture comprising one or more binding monomer(s) each independently present in an amount from about 1% (w/w) to about 90% (w/w) relative to total monomers, including about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 29% (w/w), about 30% (w/w), about 31% (w/w), about 32% (w/w), about 33% (w/w), about 34% (w/w) about 35% (w/w), about 36% (w/w), about 37% (w/w), about 38% (w/w), about 39% (w/w), about 40% % (w/w), about 41% (w/w), about 42% (w/w), about 43% (w/w), about 44% (w/w), about 45% % (w/w), about 46% (w/w), about 47% (w/w), about 48% (w/w), about 49% (w/w), about 50% % (w/w), about 51% (w/w), about 52% (w/w), about 53% (w/w), about 54% (w/w), about 55% % (w/w), about 56% (w/w), about 57% (w/w), about 58% (w/w), about 59% (w/w), about 60% % (w/w), about 61% (w/w), about 62% (w/w), about 63% (w/w), about 64% (w/w), about 65% % (w/w), about 66% (w/w), about 67% (w/w), about 68% (w/w), about 69% (w/w), about 70% % (w/w), about 71% (w/w), about 72% (w/w), about 73% (w/w), about 74% (w/w), about 75% % (w/w), about 76% (w/w), about 77% (w/w), about 78% (w/w), about 79% (w/w), about 80% % (w/w), about 81% (w/w), about 82% (w/w), about 83% (w/w), about 84% (w/w), about 85% % (w/w), about 86% (w/w), about 87% (w/w), about 88% (w/w), about 89% (w/w), to about 90% (w/w) including all values and subranges therebetween relative to total monomer. In some embodiments, the mixture comprises one or more binding monomer(s) or non-crosslinking monomer(s)) each independently present in an amount from about 30% (w/w) to about 70% (w/w) relative to total monomer.

Cross-linking (also crosslinking) agents or cross-linking monomers that impart rigidity or structural integrity to the MIP are known to those skilled in the art, and include di-, tri- and tetrafunctional acrylates or methacrylates, divinylbenzene (DVB), alkylene glycol and polyalkylene glycol diacrylates and methacrylates, including ethylene glycol dimethacrylate (EGDMA) and ethylene glycol diacrylate, vinyl or allyl acrylates or methacrylates, diallyldiglycol dicarbonate, diallyl maleate, diallyl fumarate, diallyl itaconate, vinyl esters such as divinyl oxalate, divinyl malonate, diallyl succinate, triallyl isocyanurate, the dimethacrylates or diacrylates of bis-phenol A or ethoxylated bis-phenol A, methylene or polymethylene bisacrylamide or Bismuth-acrylamide, including hexamethylene bisacrylamide lanthanide or hexamethylene bismethacrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methyl-2-isocyanatoethyl methacrylate, 1,1-dimethy 1-2-isocyanaotoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, divinyl benzene; 1,3-divinyltetramethyl disiloxane; 8,13-divinyl-3,7,12,17-tetramethyl-21H,23H-porphine; 8,13-divinyl-3,7,12,17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; divinyl tin dichloride; N,N′-methylenebisacrylamide, N,N′-bisacryloyl-1,2-dihydroxy-1,2-ethylenediamine (DHEBA); Ethylene dimethacrylate; tetraethyl orthosilicate; piperazine diacrylamide; aminophenylboronic acid (APBA); 3-Aminopropyltriethoxysilane; (3-Aminopropyl)-trimethoxysilane (APTMS); trimethylolpropane trimethacrylate (TRIM); polydimethylsiloxane (PDMS), polyacrylate, silica (SiO₂), and polyurethane (PU) and phloroglucinol; and the like.

In some embodiments, the cross-linking agent or cross-linking monomer is selected from the group consisting of divinylbenzene (DVB); N,N′-methylenebisacrylamide; N,N′-bisacryloyl-1,2-dihydroxy-1,2-ethylenediamine (DHEBA); Ethylene dimethacrylate (EDMA); tetraethyl orthosilicate (TEOS); piperazine diacrylamide; aminophenylboronic acid (APBA); 3-Aminopropyltriethoxysilane (APTES); polydopamine (PDA); (3-Aminopropyl)-trimethoxysilane (APTMS); Ethylene glycoldimethacrylate (EGDMA; trimethylolpropane trimethacrylate (TRIM); polydimethylsiloxane (PDMS); polyacrylate, silica (SiO₂); polyurethane (PU); pentaerythritol triacrylate and phloroglucinol.

In some embodiments, the cross-linking agent or cross-linking monomer is one or more monomers selected from the group consisting of ethylene glycol dimethacrylate (EGDMA), N,N′-methylenebisacrylamide, N,N′-(1,2-dihydroxyethylene)bisacrylamide, and combinations thereof.

In embodiments, the cross-linking agent or cross-linking monomer is N,N′-(1,2-dihydroxyethylene)bisacrylamide.

In embodiments, the cross-linking agent or cross-linking monomer is ethylene glycol dimethacrylate (EGDMA).

In embodiments, the cross-linking agent or cross-linking monomer is N,N′-methylenebisacrylamide.

In some embodiments, a MIP of the present disclosure is prepared by polymerizing a mixture comprising one or more crosslinking monomers, each independently present in an amount from about 1% (w/w) to about 90% (w/w) relative to total monomers, including about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 29% (w/w), about 30% (w/w), about 31% (w/w), about 32% (w/w), about 33% (w/w), about 34% (w/w) about 35% (w/w), about 36% (w/w), about 37% (w/w), about 38% (w/w), about 39% (w/w), about 40% % (w/w), about 41% (w/w), about 42% (w/w), about 43% (w/w), about 44% (w/w), about 45% % (w/w), about 46% (w/w), about 47% (w/w), about 48% (w/w), about 49% (w/w), about 50% % (w/w), about 51% (w/w), about 52% (w/w), about 53% (w/w), about 54% (w/w), about 55% % (w/w), about 56% (w/w), about 57% (w/w), about 58% (w/w), about 59% (w/w), about 60% % (w/w), about 61% (w/w), about 62% (w/w), about 63% (w/w), about 64% (w/w), about 65% % (w/w), about 66% (w/w), about 67% (w/w), about 68% (w/w), about 69% (w/w), about 70% % (w/w), about 71% (w/w), about 72% (w/w), about 73% (w/w), about 74% (w/w), about 75% % (w/w), about 76% (w/w), about 77% (w/w), about 78% (w/w), about 79% (w/w), about 80% % (w/w), about 81% (w/w), about 82% (w/w), about 83% (w/w), about 84% (w/w), about 85% % (w/w), about 86% (w/w), about 87% (w/w), about 88% (w/w), about 89% (w/w), to about 90% (w/w) including all values and subranges therebetween relative to total monomer. In some embodiments, the amount of crosslinking monomer is from about 40% (w/w) to about 70% (w/w) crosslinking monomer(s) relative to total monomer.

In embodiments, the molar ratio of binding monomers:cross-linking monomers is between 10:1 and 1:10, including between 10:1, 9:1, 8:1, 7:1, 6:1, 5:1, 4:1, 3:1, 2:1, 1:1, 1:2 1:3, 1:4, 1:5, 1:6, 1:7 1:8, 1:9, and 1:10, including all values and subranges therebetween.

In embodiments, a detection enabling tag, such as a fluorescent agent, can be incorporated into the MIP materials disclosed herein. In embodiments, the detection enabling agent can be in the substrate, on the substrate or within the MIP.

The detectable group may be any material having a detectable physical or chemical property, for example detectable by spectroscopic, photochemical, biochemical, immunochemical, fluorescent, electrical, optical or chemical means, for example, magnetic beads (e.g. DYNABEADS®); biotin, avidin, or streptavidin; radioisotopes or radionuclides (e.g., 3H, 14C, 15N, 35S, 90T, 99Tc, 111In, 125I, 131I); fluorescent labels (e.g., Texas-Red, green fluorescent protein, yellow fluorescent protein, cyan fluorescent protein, Alexa dye molecules, FITC, fluorescin and its derivatives, rhodamine and its derivatives, lanthanide phosphors, dansyl, umbelliferone etc); enzymatic labels, which may primarily be hydrolases, particularly phosphatases, esterases and glycosidases, or oxitranscription factoreductases, particularly peroxidases (e.g., alkaline phosphatase, horseradish peroxidase, beta-galactosidase, beta-lactamase, galactose oxidase, lactoperoxidase, luciferase, myeloperoxidase, and amylase); colorimetric labels such as colloidal gold colored glass or plastic (e.g. polystyrene, polypropylene, latex, etc.) beads, and nanoparticles (e.g., gold nanoparticles); chemiluminescent (e.g., luciferin, and 2,3-dihydrophthalazinediones, e.g., luminol); predetermined polypeptide epitopes recognized by a secondary reporter (e.g., leucine zipper pair sequences, binding sites for secondary antibodies, metal binding domains, epitope tags).

In embodiments, the detection enabling tag is a fluorescent dye e.g., rhodamine 110 chloride.

The monomers of the present disclosure may be polymerized by free radical polymerization, and the like. Any photoinitiators (e.g., photoinitiator for UV polymerization) or thermal free radical initiator known to those skilled in the art can be used in the preferred free radical polymerization. Examples of non-limiting suitable photoinitiators (e.g., photoinitiator for UV polymerization) and thermal initiators include: benzoyl peroxide, acetyl peroxide, lauryl peroxide, azobisisobutyronitrile (AIBN), t-butyl peracetate, cumyl peroxide, t-butyl peroxide; t-butyl hydroperoxide, bis(isopropyl) peroxy-dicarbonate, benzoin methyl ether, 2,2′-azobis(2,4-dimethyl-valeronitrile), tertiary butyl peroctoate, phthalic peroxide, diethoxyacetophenone, t-butyl peroxypivalate, diethoxyacetophenone, 1-hydroxycyclohexyl phenyl ketone, 2,2-dimethyoxy-2-phenylacetophenone, and phenothiazine, diisopropylxanthogen disulfide, 2,2′-azobis-(2-amidinopropane); 2,2′-azobisisobutyronitrile-; 4,4′-azobis-(4-cyanovaleric acid); 1,1′-azobis-(cyclohexanecarbonitrile)-; 2,2′-azobis-(2,4-dimethyl valeronitrile); and the like and mixtures thereof. In some embodiments, peroxodisulfates (ammonium peroxodisulfates (APS) or potassium peroxodisulfates (KPS)) with N,N,N,N-tetramethylethylenediamine (TEMED) are used as an initiator and catalyst that may be used in free radical polymerization to produce MIP materials of the present disclosure. In some embodiments, the initiator is selected from the group consisting of azoisobutyronitrile (AIBN), diphenyl(2,4,6-trimethylbenzoyl)phosphine oxide (TPO), 1-phenyl 1,2-propanedione (PPD), camphorquinone (CQ), and phenylbis(2,4,6-trimethylbenzoyl)phosphine oxide, and combinations thereof.

In some embodiments, a MIP material of the present disclosure is prepared by polymerizing a mixture further comprising viscosity modifier. In some embodiments, the viscosity modifier is a polymer selected from the group consisting of poly(acrylic acid), poly(ethylene oxide), poly(ethyleneimine), poly(propylene oxide), copolymers of ethylene oxide and propylene oxide, block copolymers of polyethylene oxide (PEO) and polypropylene oxide (PPO), triblock PEO-PPO-PEO polymers, and combinations thereof. In some embodiments, the viscosity modifier is poly(acrylic acid). In some embodiments, the polymer viscosity modifiers have a molecular weight (MW) ranging from about 10,000 g/mol to about 1,000,000 g/mol, including from about 10,000 g/mol, about 50,000 g/mol, about 100,000 g/mol, about 150,000 g/mol, about 200,000 g/mol, about 250,000 g/mol, about 300,000 g/mol, about 350,000 g/mol, about 400,000 g/mol, about 450,000 g/mol, about 500,000 g/mol, about 550,000 g/mol, about 600,000 g/mol, about 650,000 g/mol, about 700,000 g/mol, about 750,000 g/mol, about 800,000 g/mol, about 850,000 g/mol, about 900,000, to about 1,000,000, including all values and subranges therebetween. In some embodiments, the polymer viscosity modifiers have a molecular weight (MW) ranging from about 50,000 to about 500,000 g/mol.

In embodiments, MIP materials with an average cavity size ranging from about 10 nm to about 500 m are prepared, including from about 10 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, about 400 nm, about 420 nm, about 430 nm, about 440 nm, about 450 nm, about 460 nm, about 470 nm, about 480 nm, about 490 nm, about 500 nm, about 600 nm, about 700 nm, about 800 nm, about 900 nm, about 1 μm, about 2 μm, about 3 m, about 4 μm, about 5 μm, about 6 μm, about 7 μm, about 8 μm, about 9 μm, about 10 μm, about 11 μm, about 12 μm, about 13 μm, about 14 μm, about 15 μm, about 16 m, about 17 μm, about 18 μm, about 19 μm, about 20 μm, about 25 μm, about 30 μm, about 40 μm, about 50 μm, about 60 μm, about 70 μm, 8 about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, to about 500 μm, including all values and subranges therebetween.

In some embodiments, the MIP materials of the present disclosure are porous to facilitate mass flow in and out of the MIP material. In some embodiments, the MIP materials of the present disclosure are characterized as “macroreticular” or “macroporous,” which refers to the presence of a network of pores having average pore diameters of greater than 100 nm. In various embodiments, MIP materials with average pore diameters ranging from 100 nm to 2.4 μm are prepared. In some embodiments the average pore diameters can be about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, or about 2400 nm, including ranges between any of these values. Any suitable method for measuring porosity known in the art can be used, for example, in some embodiments, the pore diameter is measured by the Brunauer-Emmett-Teller (BET) method.

The MIP materials can also be mesoporous or include mesopores (in addition to macropores). The term “mesoporous” refers to porous networks having an average pore diameter from 10 nm to 100 nm. In some embodiments mesopore average pore diameters can be about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, or about 100 nm, including any ranges between any of these values.

In some embodiments the MIP coated articles of the present disclosure have a polymer shell with an average thickness of between 10 nm to 100 am. In some embodiments, the average thickness is about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, about 400 nm, about 420 nm, about 430 nm, about 440 nm, about 450 nm, about 460 nm, about 470 nm, about 480 nm, about 490 nm, about 500 nm, about 600 nm, about 700 nm, about 800 nm, about 900 nm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 m, about 17 μm, about 18 μm, about 19 μm, about 20 μm, about 25 μm, about 30 μm, about 40 μm, about 50 μm, about 60 μm, about 70 μm, 8 about 80 μm, about 90 m, to about 100 μm, including all values and subranges therebetween. In some embodiments, the MIP coated articles of the present disclosure have a polymer shell with an average thickness of between about 0.01 μm to about 20 μm, about 0.01 μm to about 15 μm, about 0.01 μm to about 10 μm, 0.1 μm to about 20 μm, about 0.1 μm to about 10 μm, about 0.5 μm to about 10 μm, or about 1 μm to about 5 μm, or about 2 μm to about 4 μm. In some embodiments, the thickness is measured by an inverted optical microscope equipped with a high-speed camera. In embodiments, the average polymer shell thickness has a standard deviation (SD) of about ±0.001 μm, about ±0.01 μm, about ±0.05 μm, about ±0.1 μm, about ±0.2 μm, about ±0.3 μm, about ±0.4 μm, about ±0.5 μm, about ±0.6 μm, about ±0.7 μm, about 0.8 μm, about ±0.9 μm, about ±1.0 μm, about ±1.1 μm, about ±1.2 μm, about ±1.3 μm, about ±1.4 μm, about ±1.5 μm. In embodiments, the MIP coated articles of the present disclosure have a polymer shell with an average thickness of about 5 μm. In embodiments, the MIP coated articles of the present disclosure have a polymer shell with an average thickness of about 1 μm to about 10 μm.

In embodiments, the MIP coated articles of the present disclosure have a uniformity of from about 70% to about 100%, including a uniformity from about 70%, about 71%, about 72%, about 73%, about 74%, about 75%, about 76%, about 76%, about 77%, about 78%, about 79%, about 80%, about 81%, about 82%, about 83%, about 84%, about 85%, about 86%, about 87%, about 88%, about 89%, about 90%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.5%, about 99%, to about 100%, including all subranges and values therebetween. In embodiments, the MIP coated articles of the present disclosure have a uniformity of from about 90% to about 99.9%. In embodiments, the MIP coated articles of the present disclosure have a uniformity of from about 95% to about 99.9%. In embodiments, the uniformity is at least about 95%. In embodiments, the uniformity is measured by scanning electron microscope (SEM).

In embodiments, MIP materials with a porosity ranging from about 1 m²/g to about 1000 m²/g are prepared, including from about 1 m²/g, about 5 m²/g, about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, about 110 m²/g, about 120 m²/g, about 130 m²/g, about 140 m²/g, about 150 m²/g, about 160 m²/g, about 170 m²/g, about 180 m²/g, about 190 m²/g, about 200 m²/g, about 210 m²/g, about 220 m²/g, about 230 m²/g, about 240 m²/g, about 250 m²/g, about 260 m²/g, about 270 m²/g, about 280 m²/g, about 290 m²/g, about 300 m²/g, about 310 m²/g, about 320 m²/g, about 330 m²/g, about 340 m²/g, about 350 m²/g, about 360 m²/g, about 370 m²/g, about 380 m²/g, about 390 m²/g, about 400 m²/g, about 410 m²/g, about 420 m²/g, about 430 m²/g, about 440 m²/g, about 450 m²/g, about 460 m²/g, about 470 m²/g, about 480 m²/g, about 490 m²/g, about 500 m²/g, about 510 m²/g, about 520 m²/g, about 530 m²/g, about 540 m²/g, about 550 m²/g, about 560 m²/g, about 570 m²/g, about 580 m²/g, about 590 m²/g, about 600 m²/g, about 610 m²/g, about 620 m²/g, about 630 m²/g, about 640 m²/g, about 650 m²/g, about 660 m²/g, about 670 m²/g, about 680 m²/g, about 690 m²/g, about 700 m²/g, about 710 m²/g, about 720 m²/g, about 730 m²/g, about 740 m²/g, about 750 m²/g, about 760 m²/g, about 770 m²/g, about 780 m²/g, about 790 m²/g, about 800 m²/g, about 810 m²/g, about 820 m²/g, about 830 m²/g, about 840 m²/g, about 850 m²/g, about 860 m²/g, about 870 m²/g, about 880 m²/g, about 890 m²/g, about 900 m²/g, about 910 m²/g, about 920 m²/g, about 930 m²/g, about 940 m²/g, about 950 m²/g, about 960 m²/g, about 970 m²/g, about 980 m²/g, about 990 m²/g, to about 100 m²/g, including all values and subranges therebetween. In embodiments, the porosity is measured by BET.

In embodiments, MIP materials with a crosslinking density ranging from about 10% to about 90% are prepared, including from about 10%, about 20%, about 30%, about 40%, about 50%, about 60%, about 70%, about 80%, to about 90%, including all values and subranges therebetween.

In embodiments, the MIP materials of the present disclosure have a plurality of imprinted cavities which selectively bind at least one target. In embodiments, the MIP materials are selective against random proteins and molecules in sample matrices such as saliva, blood, urine, and the like. In embodiments, the MIP materials are selective against all other viruses and proteins.

In some embodiments of MIPs of the present disclosure, the selectivity of the MIP is at least 70%, including about 70%, about 75%, about 80%, about 85%, about 90%, about 95%, about 96%, about 97%, about 98%, or about 99%, or more. Selectivity, expressed as a percentage, refers to the selectivity for the target versus other non-target species in the mixture.

The MIP coated articles according to the present disclosure are selective for the target. The selectivity of the MIP material to bind species “A” in a mixture of “A” and species “B” can be characterized by a “selectivity coefficient” using the following relationship:

$$Selectivity coefficient for A = \frac{[ {A}^{′} ] [ B ]}{[ A ] [ {B}^{′} ]}$$

where “[A]” and “[B]” refer to the molar concentration of A and B in solution, and “[A′]” and “[B′]” refer to the concentration of complexed “A” and “B” in the MIP material.

For most separations, the selectivity coefficient for the target versus other species in the mixture to be separated should be at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 70, at least about 80, at least about 90, at least about 100, at least about 200, at least about 300, at least about 400, at least about 500, including ranges between any of these values.

The term sensitivity as used herein, is the proportion of true positives tests out of all samples containing the target. In other words, the extent to which actual positives are not overlooked (so false negatives are few). The equation for sensitivity is the following:

Sensitivity=(True Positives(A))/(True Positives(A)+False Negatives(C)).

As defined herein, specificity is the percentage of true negatives out of all samples containing the target. In other words, the extent to which actual negatives are classified as such (so false positives are few). The formula to determine specificity is the following:

Specificity=(True Negatives(D))/(True Negatives(D)+False Positives(B))

For example, if the MIP material of the present invention is contacted with 100 samples containing the target, the detection of the target in 70 of the samples would provide a sensitivity of 70%. Analogously, the percent specificity would be 70% if a MIP of the present invention is contacted with 100 samples not containing the target, and correctly provides a negative result (i.e., no detection) of the target in 70 of the samples.

In some embodiments, the sensitivity of the MIP coated article for the target is at least about 70%, at least about 75%, at least about 85%, at least about 90%, at least about 95%, at least about 99%. In some embodiments, the sensitivity of the MIP coated article for the target is at least about 70%, about 75%, about 85%, about 90%, about 95%, about 99%. In some embodiments, the sensitivity of the MIP coated article for the target is about 70-99%, about 75-99%, about 80-99%, about 85-99%, about 90-99%, about 95-99%, including all ranges therebetween.

In some embodiments, the specificity of the MIP coated article for the target at least about 70%, at least about 75%, at least about 85%, at least about 90%, at least about 95%, at least about 99%. In some embodiments, the specificity of the MIP coated article for the target is at least is about 70%, about 75%, about 85%, about 90%, about 95%, about 99%. In some embodiments, the specificity of the MIP coated article for the target is at least about 70-99%, about 75-99%, about 80-99%, about 85-99%, about 90-99%, about 95-99%, including all ranges therebetween.

In embodiments, the present disclosure provides a sensor comprising the MIP coated article of the present disclosure and a transduction device, wherein when a target selectively binds to at least a portion of the imprinted cavities of the MIP coated article, the transduction device produced a signal.

In embodiments, the MIP coated article is integrated into portable microfluidic devices for rapid detection of targets, such as those described in J. R. Mejia-Salazar et al. Sensors 2020, 20, 1951; S. Krokhine et al. Colloids and Surfaces B: Biointerfaces 206 (2021) 111962; D. Zhang et al. Anal. Chem. 2018, 90, 5512-5520; and K. R. Mitchell et al., Anal Bioanal Chem. 2021 Aug. 4; 1-14, the disclosures of which are incorporated by reference herein.

### Methods

In one aspect, the present disclosure provides methods for preparing molecularly imprinted polymer (“MIP”) absorbents or materials, MIP absorbents or materials prepared by such processes, and processes utilizing the MIP absorbents or materials of the present disclosure. In some embodiments, the MIP absorbents and materials of the present disclosure are suitable for separating, extracting, or sequestering one or more biological targets, from a sample containing the biological target.

In embodiments, the present disclosure provides a method for detecting a target, comprising contacting a sensor of the present disclosure, with a fluid sample, wherein if the sample contains an amount of the target corresponding to at least the lower detection limit of the target, the transduction device provides a signal indicating the presence of the target, and if the sample contains an amount of target below the lower detection limit of the target, the transduction device does not provide a signal.

In embodiments, the present disclosure provides a method for detecting a pathogen, comprising contacting a sensor of the present disclosure, wherein the at least one target is a pathogen, with a fluid sample, wherein if the sample contains an amount of pathogen corresponding to at least the lower detection limit of the pathogen, the transduction device provides a signal indicating the presence of the pathogen, and if the sample contains an amount of pathogen below the lower detection limit of the pathogen, the transduction device does not provide a signal.

In some embodiments, the sample is any sample containing or potentially containing a target. In some embodiments, the sample is selected from the group consisting of aerosols, fluid samples, solid samples, biological samples, insects, food matrices, and environmental samples containing or potentially containing a target.

In embodiments, the present disclosure provides a method of removing one or more targets from a fluid, comprising contacting the fluid with a MIP coated article of the present disclosure, whereby at least a portion of the target in the fluid binds to the MIP coated article. In some embodiments of the methods of removing one or more targets, the fluid is water. In some embodiments, the fluid is air.

The present disclosure also provides methods of diagnosing a subject infected with a target pathogen utilizing MIPs of the present disclosure, in addition to diagnostic kits are provided. In one aspect, the present disclosure provides a kit comprising a MIP coated article or a sensor comprising a MIP coated article of the present disclosure for detecting a target pathogen, and/or unique biomarkers associated with a target pathogen. In some embodiments, the present disclosure provides methods for determining the onset, progression, or regression of an infection associated with a target pathogen in a subject, wherein a biological sample obtained from a subject is screened for said pathogen by contacting said biological sample with one or more MIP coated articles of the present disclosure having an affinity for the target virus. In one aspect, the present disclosure provides a kit comprising one or more MIPs for detecting or identifying target pathogen.

The present invention, also provides methods for detecting the presence of a target on a target area, comprising contacting the target area or a sample from the target area with one or more MIP coated articles of the present disclosure having an affinity for the target. Exemplary target areas include environmental surfaces such as hospitals, medical devices, floor sweepings, bed railings, tables, bedding, cloths, and door knobs etc.

### Biological Targets

The present disclosure provides materials, including MIP coated articles and sensors for selectively binding and detecting one or more targets. In embodiments, the MIP coated articles and sensors are used for binding and detecting a single target. In embodiments, the MIP coated articles and sensors are used for simultaneously binding and/or detecting multiple targets. In embodiments the target is a biological target. In embodiments, the target is a pathogen. In embodiments, the biological target is one or more viruses, bacteria, fungi, protozoa, and helminths (parasitic worms) and biomarkers. In embodiments of the present disclosure the biological target is typically the intact target e.g., intact microorganisms such as virus(es) or bacteria(s). However, the present disclosure also contemplates embodiments where the target is a portion of a biological target, and/or small molecule or macromolecule targets and other biomarkers. Exemplary macromolecules include proteins, including glycoproteins, peptides, polypeptides, polysaccharides, DNA, RNA, and/or antibodies associated with the biological target. In alternative embodiments, the materials, sensors, and methods of the present disclosure may be well suited for detecting hormones, and VOCs.

In embodiments, the biological target is one or more bacterium or a bacterial genus.

In embodiments, the target is one or more: Campylobacter spp., Clostridium spp., e.g., Streptococcus spp. Legionella spp., Capnocytophaga spp., Staphylococcus spp., Escherichia spp., Borrelia spp., Chlamydia spp., Helicobacter spp., Rhodococcus spp., Ehrlichia spp., non-diphtheria Corynebacterium spp., spotted fever group Rickettsia spp., Anaplasma spp., Tropheryma spp., Vibrio spp., Bartonella spp., Aerococcus spp., Wolbachia spp., Simkania spp., Actinobaculum spp., Parachlamydia spp., Waddlia spp., Alloscardovia spp., and Neoehrlichia spp. Listeria spp. and Salnonella spp.

In embodiments, the target is one or more of the following bacteria: Campylobacter jejuni, Clostridium difficile, Streptococcus bovis group, Legionella pneumophila, Capnocytophaga canimorsus, Staphylococcus aureus, Escherichia coli, Borrelia burgdorferi, Chlamydia pneumoniae, Helicobacter pylori, Rhodococcus equi, Ehrlichia chaffeensis, Corynebacterium amycolatum, R. africae, R. helveticae, R. slovaca, R. mongolotimonae, Anaplasma phagocytophilum, Tropheryma whipplei, Vibrio cholerae e.g., Vibrio cholerae O139, Bartonella henselae, A. urinae, A. sanguinicola, Simkania negevensis, Actinobaculum schaalii, Parachlamydia acanthamoebae, Waddlia chondrophila, Alloscardovia omnicolens, and Neoehrlichia mikurensis.

In embodiments, the target is one or more of the following: Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Sarcina spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In embodiments, the biological target is Campylobacter jejuni, enterotoxigenic Escherichia coli, Shigella spp., Vibrio cholerae, Aeromonas spp., enterotoxigenic Bacteroides fragilis, Clostridium difficile or Cryptosporidium parvum

In embodiments, the target is Escherichia coli.

In embodiments, the target is Escherichia coli O157:H7.

In embodiments, the target is Sarcina lutea.

In embodiments, the biological target is one or more fungal pathogens. In embodiments, the fungal pathogen is selected from the group consisting of Candida spp., Cladosporium spp., Aspergillus spp., Penicillium spp., Alternaria spp., Pleospora spp., Fusarium spp. In embodiments, the fungal pathogen is Candida lusitaniae, Candida parapsilisis, or Candida albicans

In embodiments, the biological target is one or more protozoa. The protozoa that are infectious to humans can be classified into four groups based on their mode of movement: Sarcodina—the ameba, e.g., Entamoeba; Mastigophora—the flagellates, e.g., Giardia, Leishmania; Ciliophora—the ciliates, e.g., Balantidium; and Sporozoa—organisms whose adult stage is not motile e.g., Plasmodium, Cryptosporidium.

In embodiments, the biological target is one or more helminths. There are three main groups of helminths that are human parasites: flatworms (platyhelminths)—these include the trematodes (flukes) and cestodes (tapeworms); thorny-headed worms (acanthocephalins); and roundworms (nematodes).

In embodiments, the biological target is one or more viruses or viral genus. In some embodiments, the target virus is a norovirus, rotavirus, adenovirus, astrovirus, influenza virus, coronavirus, respiratory syncytial virus, human immunodeficiency virus (HIV), human T lymphotrophic virus (HTLV), rhinovirus, hepatitis A virus, hepatitis B virus, Epstein Barr virus, West Nile virus, zika virus, ebola virus, or human parainfluenza viruses (HPIV) respiratory virus. In embodiments, the virus is rotavirus, including strains of rotavirus e.g., G1 or G12. In embodiments, the virus is an influenza virus, such as influenza A or B. Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). Current subtypes of influenza A viruses that routinely circulate in people include: A(H1N1) and A(H3N2). In embodiments, the influenza B virus is B/Yamagata or B/Victoria.

In embodiments, the MIP coated articles of the present disclosure the target is one or more Coronaviruses. Coronaviruses are named for the crown-like spikes on their surface. There are four main sub-groupings of coronaviruses, known as alpha, beta, gamma, and delta. Seven coronaviruses that can infect people are: 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19). People around the world commonly are infected with human coronaviruses HCoV-229E, NL63, OC43, and HKU1.

In embodiments the MIPs of the present disclosure are capable of selectively binding to intact SARS-CoV-2, to an attenuated SARS-CoV-2, or to a portion of SARS-CoV-2 (e.g., SARS-CoV-2 spike glycoprotein).

In embodiments, the target is one or more hormones or volatile organic compounds (VOCs). VOCs are compounds that have a high vapor pressure and low water solubility. Target VOCs include human volatile organic compounds (VOCs) detected in exhaled breath, and electronic nose applications. See e.g., Berna et al. ACS Infectious Diseases 2021 7 (9), 2596-2603, which is incorporated by reference herein.

### EXAMPLES

### Example 1: Preparation of Core-Shell Microspheres and Shell Thickness Optimization

### Chemicals and Materials

The monomers acrylamide (AAM), methacrylic acid (MAA), methylmethacrylate (MMA) and N-vinylpyrrolidone (VP), the cross-linker dihydroxyethylene-bisacrylamide (DHEBA), the initiator 2,20-azobis(isobutyronitrile) (AIBN), dimethyl sulfoxide (DMSO), and Rhodamine 110 chloride were purchased from Sigma-Aldrich. All the reagents were of the highest purity available and used without further processing. A non-pathogenic Escherichia coli OP50 bacteria strain was obtained from Caenorhabditis Genetics Center (University of Minnesota, USA) and used as the template. Carboxyl-terminated magnetic polystyrene microparticles (19±0.5 μm, Spherotech Inc., Product No.: CM-200-10, USA) were used as core particles.

### Polymer Synthesis

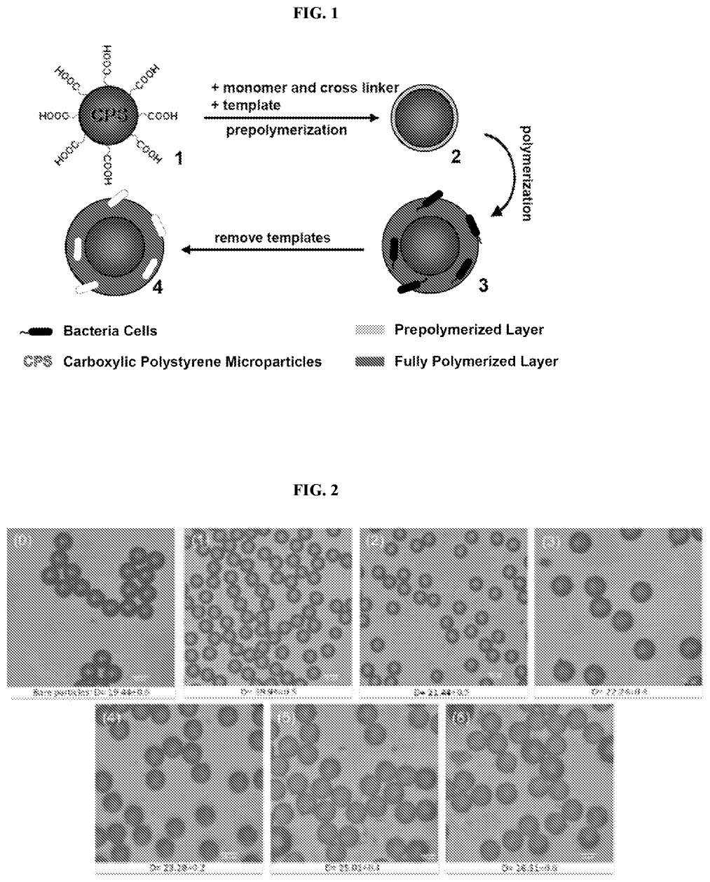

The experimental process of molecularly imprinted polymers on microspheres is summarized in FIG. 1. The monomer combination in Table 1 was mixed in the presence of the cross-linker, solvent, and initiator in a 1.5 ml microcentrifuge tube. Then, the mixture was ultrasonicated for 2 min to remove dissolved gasses. Carboxylic-terminated polystyrene microspheres (CPS) were then dispersed in the above solution in concentration of 10⁵/mL. Finally, E. coli OP50 suspension (50 μL, 10⁶ cells/mL) was added into the microtube and shaken for 15 min.

TABLE 1
Recipe used for molecular imprinting.

| Chemicals | | Quantity/concentration |
|---|---|---|
| Functional | Acrylamide | 13 mg; 101.6 μmol |
| Monomers | Methacrylic acid | 10.6 μl; 65.8 μmol |
| | Methylmethacrylate | 6 μl; 31.2 μmol |
| | N-vinylpyrrolidone | 6.3 μl; 31.2 μmol |
| Crosslinker | Dihydroxyethylene | 47 mg; 130.4 μmol |
| | bisacrylamide | |
| Solvent | Dimethyl sulfoxide | 400 μl |
| Initiator | 2, 20-azobis(isobutyronitrile) | 1 mg; 3.4 μmol |

Subsequently, a two-step temperature-dependent polymerization method was carefully designed to control the imprinting polymerization as well as the thickness of imprinted shell at the surface of microspheres. Prepolymerization (first step) was performed and subsequently polymerization was completed (second step) at different dispersion times and temperatures according to Table 2, using an air circulated gravity convection oven (Thermo Fisher Scientific, Heratherm™, Germany).

The hypothesis of using two polymerization steps relies on the use of slow polymerization rate to allow for enough time to deposit the polymer. Briefly, at the pre-polymerization step, low temperature was used to coat a thin oligomer layer on the CPS surface, allowing for the formation of strong hydrogen-bonding interactions between the surface carboxyl groups and functional monomers. These interactions drive the imprinting precursors to assemble on CPS spheres, and thus, surface pre-polymerization occurred. After the formation of thin oligomer shells, the imprinting polymerization process was completed at high temperature. The resulting polymer was preferentially nucleated and grew on the surface of CPS spheres, resulting in the formation of uniform core-shell microspheres.

The resultant core-shell PS-MIP microspheres were isolated from the solution using an external magnetic field and the supernatant was discarded from the flask. MIP coated microspheres were sequentially washed with methanol-acetic acid solution (9:1, v/v), methanol and deionized water.

TABLE 2
Two-step temperature-rising polymerization at
different temperatures and times.

| | First Step | | Second Step | |
|---|---|---|---|---|
| Sample | T (° C.) | t (h) | T (° C.) | t (h) |
| a | 40 | 6 | 50 | 6 h |
| b | 40 | 6 | 50 | 8 h |
| c | 40 | 6 | 50 | 10 h |
| d | 50 | 4 | 60 | 2 h |
| e | 50 | 4 | 60 | 3 h |
| f | 50 | 4 | 60 | 4 h |

The morphology of the bacteria-imprinted PS microparticles was observed using a scanning electron microscopy (Quanta 3D FEG Thermofisher, USA). An inverted optical microscope (Bioimager, Canada) equipped with a high-speed camera (FASTEC IL3, Canada) was used for particles' size distribution and shell thickness determination. A fluorescent microscope (ZEISS, Germany) was used to visualize the shell integrity. Particle size measuring was carried out using the image processing technique with ImageJ and a developed MATLAB code.

To further show the applicability of integrating colorimetric imaging of the coated microparticles, a fluorescent dye Rhodamine 110 chloride was mixed with the pre-polymer solution and used to prepare a thin layer of fluorescent shell on the microparticles. PS-non-imprinted polymers (NIP) microspheres were similarly synthesized in the absence of OP50 templates for comparison purposes.

### Results

SEM imaging showed that the surface of PS microparticles became rougher after polymer coating compared to the bare particles.

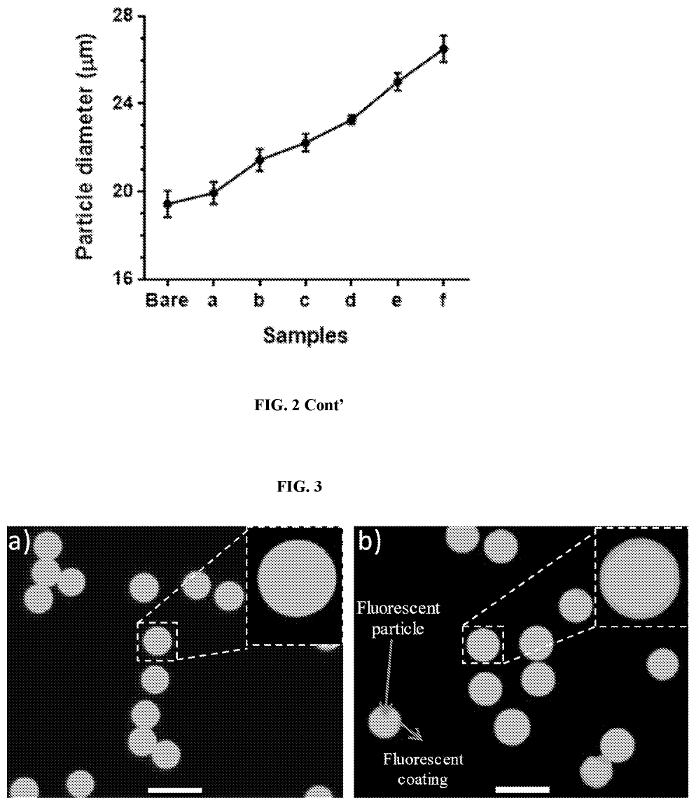

FIG. 2 shows optical microscopy images of MIP-coated microparticles. (0) shows uncoated CPS and (1) to (6) show coated particles using two-step temperature-rising polymerization method with the recipes (a-f) in Table 2.

FIG. 2 also shows the diameter of the non-coated and coated microparticles with the recipes (a-f) in Table 2. The obtained shell thickness varied from around 0.5 μm to nearly 7 μm as shown in the Table below.

| | First Step | | Second Step | | Shell thickness |
|---|---|---|---|---|---|
| Sample | T (° C.) | t (h) | T (° C.) | t (h) | (mean ± SD, um) |
| a | 40 | 6 | 50 | 6 h | 0.5 ± 0.1 |
| b | 40 | 6 | 50 | 8 h | 1.94 ± 0.3 |
| c | 40 | 6 | 50 | 10 h | 2.64 ± 0.5 |
| d | 50 | 4 | 60 | 2 h | 3.58 ± 0.4 |
| e | 50 | 4 | 60 | 3 h | 4.89 ± 0.6 |
| f | 50 | 4 | 60 | 4 h | 6.03 ± 1.1 |

At constant polymerization temperatures, the shell thickness increased with increasing the polymerization time. Similarly, increasing the polymerization temperatures promoted more polymer coating on the surface. This may be attributed to the increased polymer solution viscosity at higher temperatures and longer polymerization time, promoting the formation of strong hydrogen bonding.

FIG. 3 depicts the fluorescent images of the bare and coated microparticles, illustrating the potential of adding a fluorescent coating for better visualization. Integrating a fluorescent dye will help in using the developed particles for colorimetric detection and quantification of bacteria and other molecules.

### Example 2: Preparation of E. Coli Imprinted Core-Shell Microspheres

### Bacteria Culturing

All experiments involving bacterial cultures were executed in a biosafety level 2 laboratory, designed, and managed in accordance with safety regulations. The E. coli OP50 strain was cultured at 37° C. for 24 h in Luria Broth (LB) agar. Similarly, colonies were suspended in 30 mL LB liquid growth medium (10 g bacto-tryptone, 5 g bacto-yeast, and 5 g NaCl in 1 L distilled water) and cultured at 37° C. overnight in a thermal shaker incubator. After centrifugation at 7000 g for 15 min, the supernatant was removed. The pellet was resuspended into fresh phosphate buffer by shaking for 1 min. These procedures were repeated three times.

### Preparation of Bacteria Imprinted Core-Shell Microspheres

The thickness of the prepared shell in sample (e) of Example 1 was in the appropriate range for OP50 cells imprinting which are around 3 to 4 μm in length. Therefore, sample (e) was considered for investigating the bacteria imprinting procedures.

The bacteria imprinted polymer layer was synthesized at the particles surface by mixing the particles and 40 μL of 10⁸ CFU/mL E. coli OP50 solution with the prepolymer solution of sample (e) from Example 1.

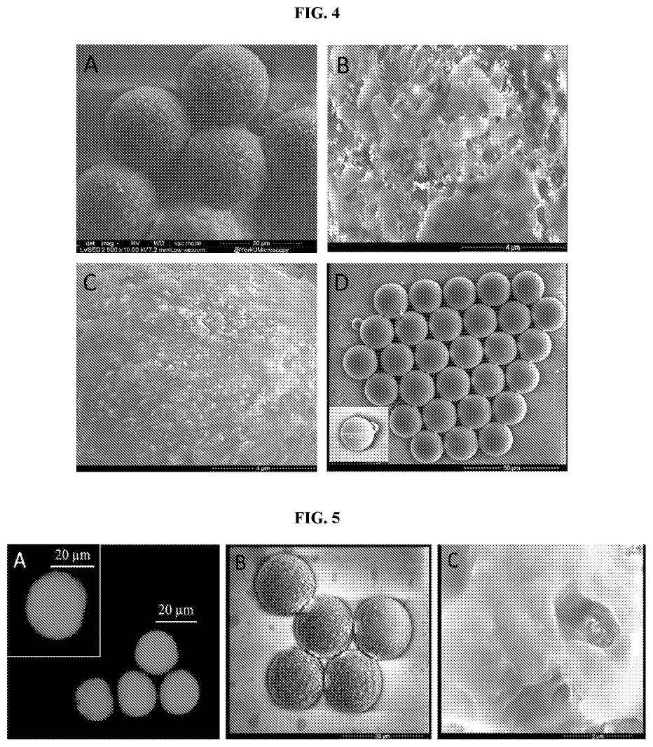

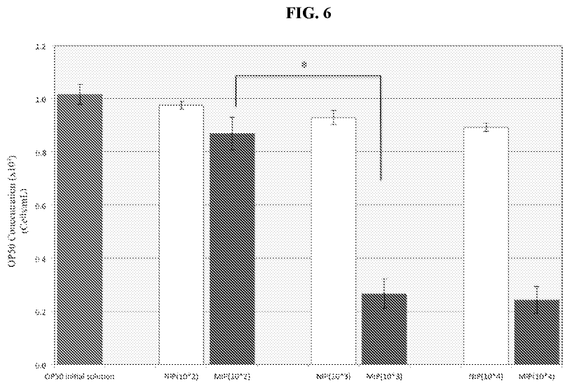

Nonimprinted core-shell microspheres (PS-NIP) were synthesized under identical conditions in the absence of OP50 templates using cell-free broth for comparison with the bacteria imprinted polymers. Nonimprinted microspheres (FIG. 4(D)) had a smooth and clear layer of polymer coating.

### Results

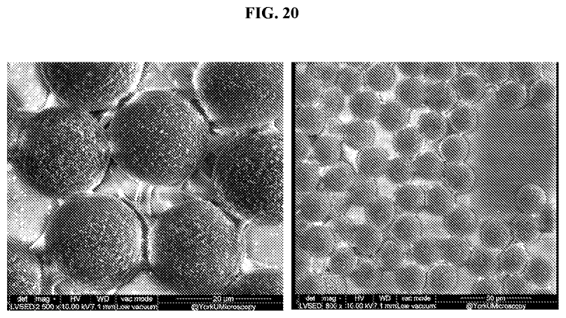

FIG. 20 shows the SEM images of the bare, uncoated particles. FIG. 4(A) and FIG. 4(B) show imprinted core-shell particles, and imprinted polymer shell before template removal. FIG. 4 (C) shows non-imprinted polymer (NIP) coated particles, (D) NIP core-shell microspheres (PS-NIP). For the bacteria-imprinted particles, during the polymerization, OP50 cells are embedded into the cross-linking polymer shells. FIG. 4(B) clearly demonstrated that the bacteria cells were entrapped in the polymer complex layer, highlighting the applicability of our technique for bacteria imprinting. Moreover, the MIP particles exhibited nearly the same morphological characteristics as those of nonimprinted core-shell microspheres but with a very rough surface with rod shapes. Thus, these figures evidently confirm that the observed bacilli shapes on the MIP coated surface (FIG. 4(B)) are bacteria OP50 cells.

The template removal was carried out using an acidic solution of methanol and acetic acid (8:1) for 30 s.

FIG. 5(A) shows a fluorescent image of MIP coated particles after template removal. FIG. 5(B) shows SEM images of the MIP coated particles surface after OP50 removal and FIG. 5(C) shows a complementary cavity on the MIP shell after template removal.

As anticipated, the extraction reaction efficiently removed the imprinted cells from the shell and thereby exposed bacilli-like surfaces complementary to E. coli. The formed cavities can be seen in FIG. 5(C) which have been formed after bacteria template removal.

### Example 3: Binding Performance of E. coli Molecularly Imprinted Core-Shell Microparticles

After the removal of OP50 templates, the molecular-recognition properties of PS-NIP and MIP microspheres were evaluated by the measurement of the rebinding capacity to E. coli OP50. E. coli adsorption and uptake ratio of bacteria cells was determined by measuring the difference between the total amount of bacteria and the residual amount in the solution phase.

### Rebinding Protocol

A mixture of E. coli OP50 (10⁴ cells/mL) and the imprinted microspheres in three different concentrations of 10²/mL, 10³/mL and 10⁴/mL were incubated at room temperature for 30 min, after which the microspheres were precipitated by gravity. After incubation for 20 min, core-shell microspheres were magnetically removed from the solution phase. The number of bound bacteria was determined by measuring the difference between the total amount of bacteria and the residual amount in the solution phase using dilution plating method. For this, the solution phase (which contains residual concentration of bacteria after particle removal) was diluted serially in sterile LB. 100 microliter of the solution was pipetted into a dilution tube containing 900 microliter of sterile LB. This tube was vortexed for approximately 5 s. After vortexing, 100 microliter of this volume was removed and placed into a second dilution tube containing 900 microliter of sterile LB. This process was repeated exactly until there was sufficient diluting of the sample. 100 microliter of the final diluted sample was plated on agar plates. All experiments were carried out with three replicates and the total number of colonies (CFU/ml) was counted after 20 h of incubation at 37° C. to determine the actual concentration of residual bacteria cells in the suspension after OP50 removal using imprinted particles.

### Results

Static adsorption data were collected at initial concentration of 1×10⁴ CFU mL⁻¹ for E. coli OP50 bacteria cells. Capturing process was done using different amounts of MIPs to observe the dose dependent bacteria adsorption. FIG. 6 shows the bacteria cells uptake ratio for different concentrations of MIPs and NIPs. The MIPs exhibited a significantly higher adsorption capacity than that of the NIPs across the tested concentration range (p<0.05). These results were mainly due to the specific adsorption of imprinted sites, leading to superior adsorption of OP50 cells. A trace of bacteria was absorbed on the NIPs simply based on non-specific adsorption.

The binding capacity also increased with increasing MIP concentrations from 10²/mL to 10³/mL. In other words, the obtained uptake ratio for 10²/mL MIP concentration was 14% which increased to 74% for 10³/mL MIP concentration (FIG. 6). This result is mainly due to the increment of binding sites when more concentration of MIPs is used for bacteria capturing.

Further increasing MIP concentration from 10³/mL to 10⁴/mL, did not cause a significant difference in the uptake ratio of bacteria cells (p>0.05). This could be because of the fact that binding sites will be partially blocked due to particles agglomeration in high concentration of MIP and the accessible sites for bacteria binding remains almost unchanged.

### Example 4: Fluorometric Detection and Binding Performance of E. coli Imprinted Core-Shell Microparticles

In a non-limiting example of bio-detection provided by the present disclosure, E. coli imprinted MIP-coated particles were prepared according to the protocol described in Example 1, MIP having cavities that are complementary in shape and size to the E. coli cells. The cavities located in the MIP shell allow for the excitation of the fluorescent core, causing a fluorescent expression. Thus, the greater the bacteria amount in these cavities, the less fluorescent expression passing through the cavities.

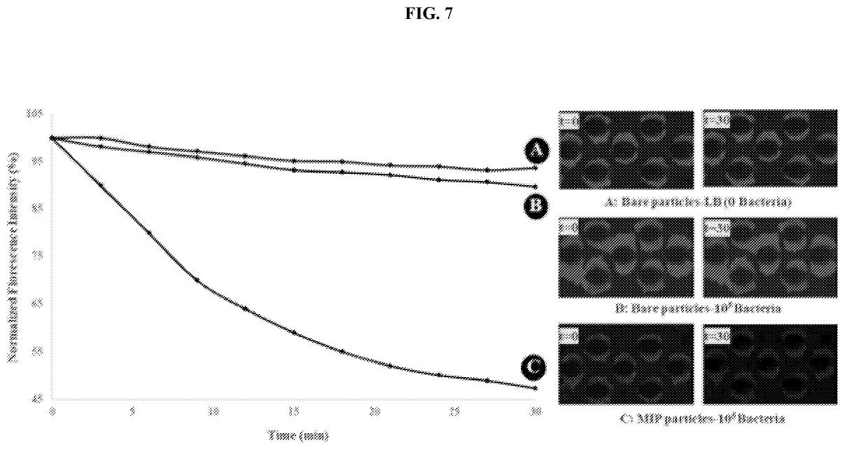

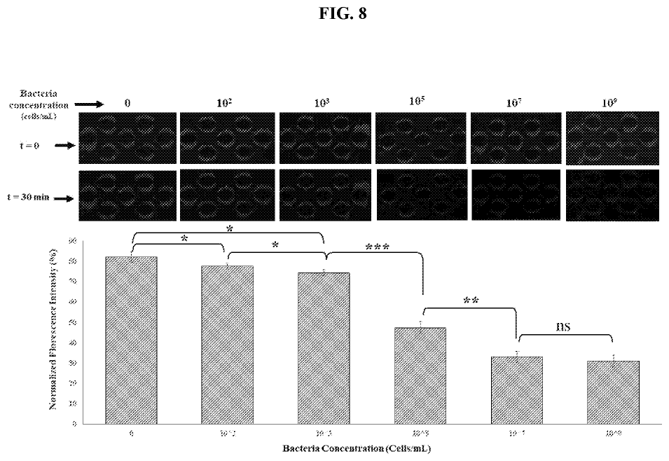

The E. coli imprinted beads were loaded to a microfluidic device at the concentration of 500 beads/μL. Then, E. coli OP50 solutions of different concentrations of 0, 10², 10³, 10⁵, 10⁷, and 10⁹ CFU/mL were prepared in LB. A syringe pump was used to flow the bacteria over the particles at a flow rate of 0.2 mL/min for 30 minutes. At first, two control experiments were performed as a positive and negative control. The negative control experiments were achieved by exposing bare (uncoated) particles without any MIP coated layer to a solution of LB (0 concentration of bacteria) and LB with 10⁵ CFU/ml of E. coli OP50, as shown in FIG. 7. In addition, the positive control experiments were performed using MIP particles in the absence of bacteria (0 concentration of bacteria), as shown in FIG. 8.

The normalized fluorescent expression of the particles at different concentrations 00, 10², 10³, 10⁵, 10⁷, and 10⁹ CFU/ml in LB. As could be seen, the MIP-coated particles at 0 concentration of bacteria are exhibiting a slight decrease in their fluorescent intensity over time, indicating a possible 10-15% decrease due to photobleaching. The results after running bacteria in different concentrations showed a reduction in fluorescent expression over time in all concentrations. For 10², concentration of bacteria compared to control sample (zero concentration), a meaningful florescent intensity reduction was observed (p value: 0.04). This result showed the limit of detection (LOD) of ˜10² cells/mL for our sensor.

In addition, a significant reduction in normalized fluorescent intensity at concentration of 10⁵ cfu/mL was reported, providing that the optimum capturing efficiency for MIP coated particles can be obtained at concentration of 10⁵ cfu/mL.

### Example 4: Preparation of Sarcina Lutea Imprinted Core-Shell Microspheres and

### Preparation of Sarcina Lutea Imprinted Core-Shell Microspheres

Sarcina Lutea imprinted core-shell microspheres were prepared according to the protocol in Example 2 using Sarcina Lutea as the template instead of E. coli.

### Rebinding Experiment

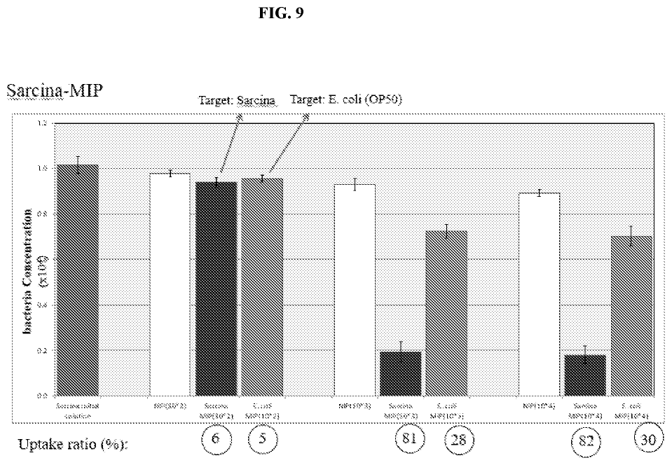

A re-binding performance test was carried according to the protocol outlined in Example 3 to evaluate the Sarcina Lutea uptake efficiency of Sarcina Lutea imprinted core-shell microspheres. As shown in FIG. 9 the uptake ratio for 10²/mL MIP concentration was % 6, increasing MIP concentration to 10³/mL and 10⁴/mL increased the uptake ratio to % 81% and 82%, respectively. The Sarcina Lutea MIPs also exhibited a significantly higher uptake ratio for Sarcina Lutea across the MIP concentration range of 10³/mL and 10⁴/mL compared to non-imprinted microsphere controls and compared to E. coli imprinted core-shell microspheres.

### Example 5: Preparation of T4 Phage Imprinted Core-Shell Microspheres and Binding Performance Tests

### Preparation of T4 Phage Imprinted Core-Shell Microspheres

T4 phage imprinted core-shell microspheres were prepared according to the protocol in Example 2 using conditions (a) in Table 2 and T4 phage as the template instead of E. coli.

### Dose-Dependent Binding Test

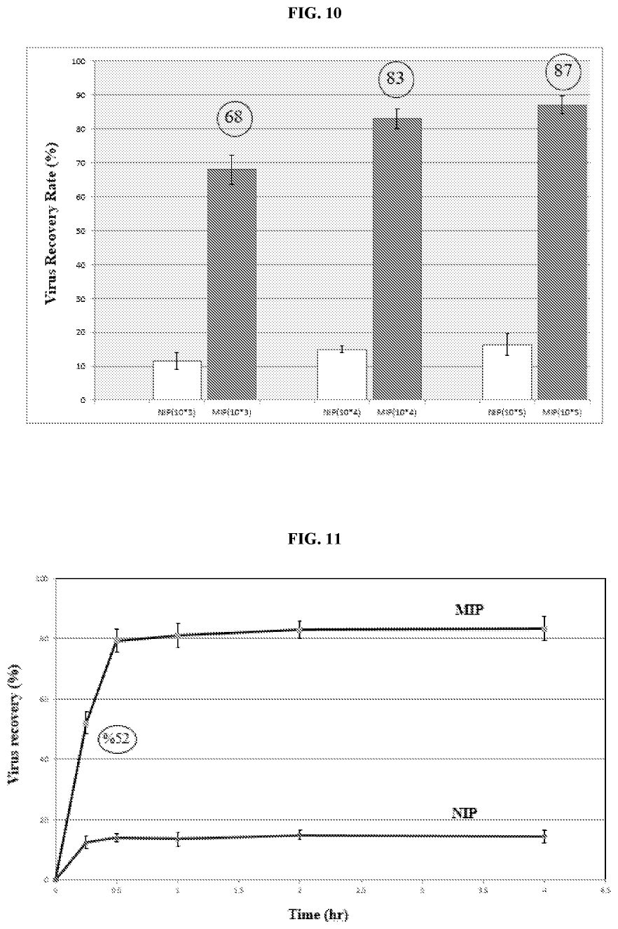

A dose-dependent binding performance test was carried out where a mixture of T4 phage cells (10³ PFU/mL) and the imprinted microspheres in three different concentrations of 10³/mL, 10⁴/mL and 10⁵/mL were incubated at room temperature for 2 hours, after which the microspheres were precipitated by gravity. Non-imprinted particles in the same concentrations as controls. The virus recovery (%) was calculated based on the following equation. The results are depicted in FIG. 10.

$$Virus Recovery ( \% ) = \frac{Initial phage titer - Supernatant phage titer}{Inital phage titer} \times 100$$

As shown in FIG. 10, the MIP microspheres showed high selectivity for the T4 phage resulted in significantly higher virus recovery compared to non-imprinted controls. The binding capacity also increased with increasing MIP concentrations from 10³/mL to 10⁵/mL.

### Time-Dependent Binding Test

A time-dependent binding performance test was carried out where a mixture of T4 phage cells (10³ PFU/mL) and the imprinted microspheres or non-imprinted microsphere control at a concentration of 10⁴/mL were incubated at room temperature for 15 min, 30 min, 45 min, 1 h, 2 h, 3 h, 6 h, 9 h. The results are shown in FIG. 11.

As shown in FIG. 11, the MIP microspheres showed high selectivity for the T4 phage reaching ˜80% virus recovery after 0.5 hours compared to non-imprinted MIPs which showed <20% virus recovery.

### Concentration-Dependent Binding Test

A time-dependent binding performance test was carried out where a mixture of T4 phage cells at different concentrations (10², 10³, and 10⁴, PFU/mL) and the imprinted microspheres or non-imprinted microsphere control at a concentration of 10⁴/mL were incubated at room temperature.

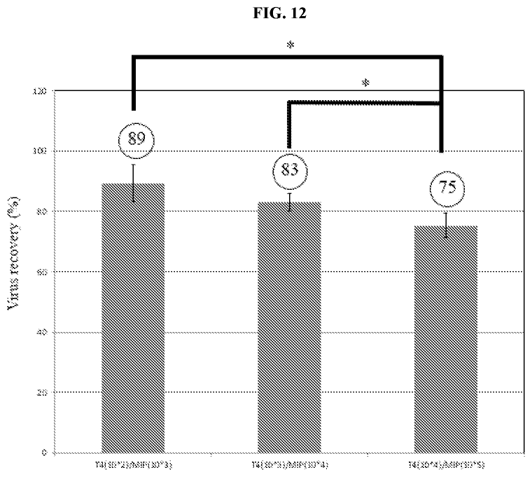

As shown in FIG. 12, the MIP microspheres can capture about 90% of virus cells even in solutions containing low concentrations of target.

These results demonstrate that this MIP coating method is appropriate and compatible for the detection of both bacteria and virus targets.

### Colorimetric Detection of Virus Targets

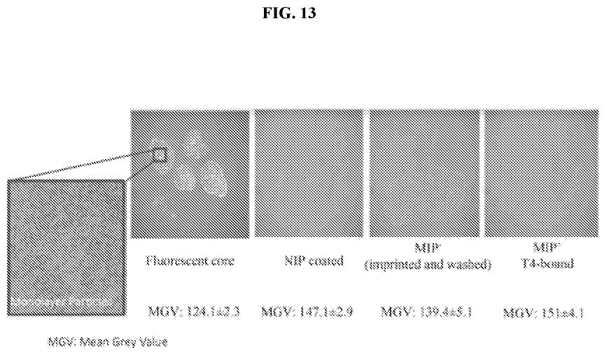

As shown in FIG. 13, the T4 phase cell-bound MIPs achieved a higher MGV value of 151±4.1 as compared to the unbound MIP microspheres control (MGV: 139.4±5.1). The increase in MGV value corresponds to a reduced exposure of the MIP fluorescent core upon T4 binding, demonstrating the binding/detection ability of the MIP.

### Example 6: Preparation of MIP Coated Metallic Microwires and Optimization

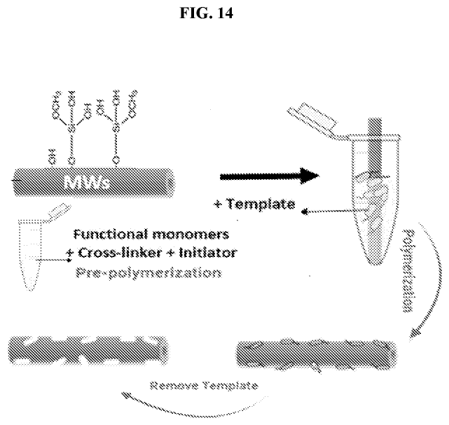

Initial studies were carried out to evaluate functionalization of surface-treated electrically conductive stainless steel (SS) microwires with MIPs made with 1-4 functional monomer types: monomers acrylamide (AAM), methacrylic acid (MAA), methylmethacrylate (MMA) and N-vinylpyrrolidone (VP), and a crosslinking monomer for application in virus and bacteria imprinting. The diameter of bare non-surface treated ss wire is: 125.3±0.5 μm. A schematic illustration of the experimental process of molecularly imprinted polymers on microwires is shown in FIG. 14.

First, a statistical model for controlled coating of different combinations of 1 functional monomer-systems were developed, varying coating conditions, temperature and time as shown below. The thickness of the coating, as well as the uniformity and morphological structure of the coating (stability, retention) were evaluated. The results are summarized in Table 3 below, and FIG. 17.

TABLE 3
Summary table of Taguchi results
MIP formulation/Coating conditions (Time & Temp)

| | Cross-linker | | | |
|---|---|---|---|---|
| Functional | (Monomer's molar | | | Temperature |
| Monomers | ratio to cl's) | Solvent | Time | (° C.) |
| 1. MAA | 1. 1:1 | 1. Acetonitrile | 1. 4 h | 1. 60 |
| 2. AAM | 2. 1:2 | 2. DMSO | 2. 7 h | 2. 70 |
| 3. VP | 3. 1:4 | 3. PBS | 3. 12 h | |
| 4. MMA | 4. 1:6 | 4. Toluene | | |

| Sample | Monomer | Cross | Solvent | Initiator | Time | Temp | |
|---|---|---|---|---|---|---|---|
| # | (μL) | linker (μL) | (mL) | (mg) | (h) | (° C.) | pH |
| 1 | MAA | EGDMA | Acetonitrile | 38.4 | 7 | 60 | 3.97 |
| | 253.11 | 566.34 | 3.2778 | | | | |
| 2 | MAA | EGDMA | DMSO | 25.6 | 7 | 60 | 6.93 |
| | 168.74 | 755 | 3.69496 | | | | |
| 3 | MAA | EGDMA | PBS | 12.8 | 12 | 70 | — |
| | 84.37 | 755.1 | 3.35788 | | | | |
| 4 | MAA | EGDMA | Toluene | 12.8 | 12 | 70 | 5.97 |
| | 84.37 | 1130 | 4.85748 | | | | |
| 5 | AAM | EGDMA | DMSO | 38.4 | 12 | 70 | — |
| | 213.24 | 566.34 | 3.11832 | | | | |
| 6 | AAM | EGDMA | Acetonitrile | 25.6 | 12 | 70 | — |
| | 142.16 | 755 | 3.58864 | | | | |
| 7 | AAM | EGDMA | Toluene | 12.8 | 7 | 60 | 7.08 |
| | 71.08 | 755.1 | 3.30472 | | | | |
| 8 | AAM | EGDMA | PBS | 12.8 | 7 | 60 | 6.75 |
| | 71.00 | 1130 | 4.80432 | | | | |
| 9 | VP | EGDMA | PBS | 38.4 | 7 | 70 | 6.73 |
| | 321 | 566.34 | 3.54936 | | | | |
| 10 | VP | EGDMA | Toluene | 25.6 | 7 | 70 | — |
| | 214 | 755 | 3.876 | | | | |
| 11 | VP | EGDMA | Acetonitrile | 12.8 | 12 | 60 | 6.39 |
| | 107 | 755.1 | 3.4484 | | | | |
| 12 | VP | EGDMA | DMSO | 12.8 | 12 | 60 | 8.01 |
| | 107 | 1130 | 4.948 | | | | |
| 13 | MMA | EGDMA | Toluene | 38.4 | 12 | 60 | 6.25 |
| | 321 | 566.34 | 3.5493625.6 | | | | |
| 14 | MMA | EGDMA | PBS | 25.6 | 12 | 60 | 7.27 |
| | 214 | 755 | 3.876 | | | | |
| 15 | MMA | EGDMA | DMSO | 12.8 | 7 | 70 | 4.13 |
| | 107 | 755.1 | 3.4484 | | | | |
| 16 | MMA | EGDMA | Acetonitrile | 12.8 | 7 | 70 | 4.96 |
| | 107 | 1130 | 4.948 | | | | |

| Sample # | Thickness | SD | Uniformity % | Stability |
|---|---|---|---|---|
| 1 | 131.87 | 0.34 | 99.73 | Yes |
| 2 | 128.38 | 0.71 | 99.44 | No |
| 3 | 130 | 6.72 | 94.83 | No |
| 4 | 126.8 | 0.85 | 99.32 | Yes |
| 5 | 126.52 | 0.76 | 99.39 | — |
| 6 | 127 | 0.7 | 99.44 | Yes |
| 7 | 128 | 0.9 | 99.29 | No |
| 8 | 132.77 | 3.42 | 97.42 | No |
| 9 | 134.1 | 15 | 88.81 | No |
| 10 | 125.26 | 0.67 | 99.46 | No |
| 11 | 131.12 | 2.71 | 97.93 | No |
| 12 | 132.53 | 3.07 | 97.68 | No |
| 13 | 125 | 0.3 | 99.76 | No |
| 14 | 128.38 | 0.71 | 99.44 | No |
| 15 | 125.93 | 0.7 | 99.44 | No |
| 16 | 126.4 | 0.45 | 99.64 | — |

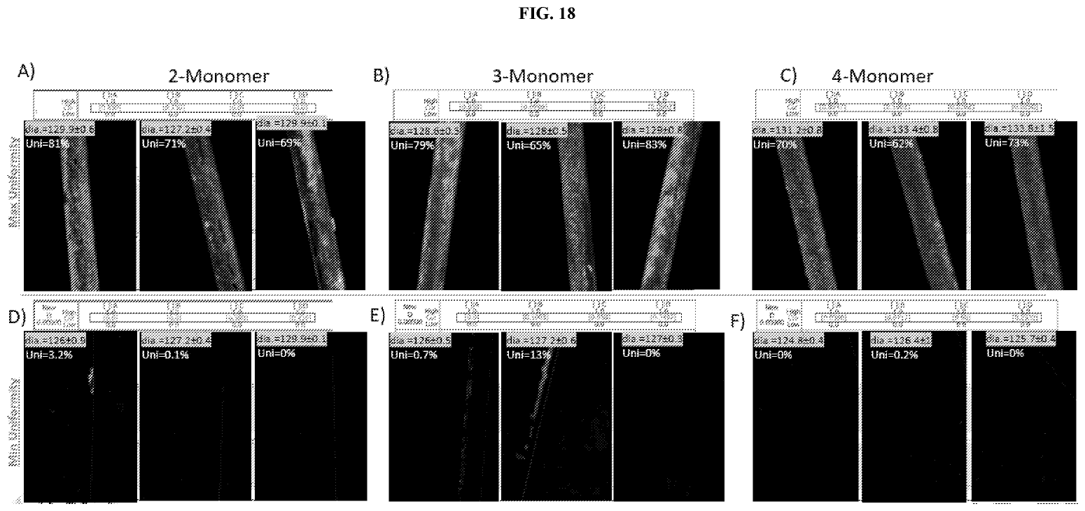

Using this model optimal recipes using MIP coatings made MIPs made with 1-4 functional monomer types (MAA, AAM, VP, and MMA), and a crosslinking monomer was identified and used to synthesize uniform MIP coatings with specific thickness and coverage uniformity on SS wires (see FIG. 17 and FIG. 18 where [A] is MAA, [B] is AAM, [C] is VP and [D] is MMA).

### Example 7: Transduction and Capturing Ability of MIP Wires

### Synthesis

To develop a ˜2 μm thick and uniform MIP coating on 125.3±0.5 μm diameter stainless-steel (SS-MWs), the free radical polymerization method was used as shown in FIG. 14. To enhance SS-MIP binding, MW surfaces were functionalized with hydroxyls using 2M sulfuric acid solution for 2 h and silanes using tetraethoxysilane (TEOS)-methanol-water (2-8-1 vl %) for 30 mins. MIP pre-polymer solution consisted of AAM (417 μmol), MAA (1.5 mmol), MMA (245 μmol), VP (31.2 μmol), EGDMA (3 mmol), and AIBN (200 μmol) dispersed in ac-etonitrile containing Rhodamine 110 (a fluorescent dye). First, pre-polymerization was carried out at 65° C. for 30 min to increase the cocktail viscosity for homogeneous dispersion of template bacteria in the next step. E. coli OP50 solution (1:4 v/v % to the pre-polymer solution, 109 CFU/mL) was then suspended in the pre-polymer by shaking for 2 mins. Surface-treated SS-MWs were then immersed in 1 mL of the MIP pre-polymer solution, and polymerization was performed in the optimized condition of 65° C. for 11 hr. The MWs were washed with methanol-acetic acid (9/1 v/v %), methanol, and DI water and dried for fluorescent imaging.

The rebinding assay was performed by incubating the MIP-MWs with bacteria suspensions of different counts for 30 mins and plate-counting the concentration of bacteria pre- and post-incubation. Briefly, MIP-MWs were installed perpendicular to a microfluidic channel for resistance-based detection of bacteria. A DC current sweep from 10 nA to 1 μA was applied for the resistance measurements between the terminal and ground MIP-MWs, while interwire voltage was measured using a DC electrical source at pre, concurrent to and post-exposure to bacteria. The pre- and post-measurements were determined by passing pure 3 ppm NaCl solution as an electrolyte through the channel, which provided the baseline resistance of each device. The post-exposure resistance, after bacteria capturing by MIPs, was normalized to the baseline resistance and used for analysis.

### Results

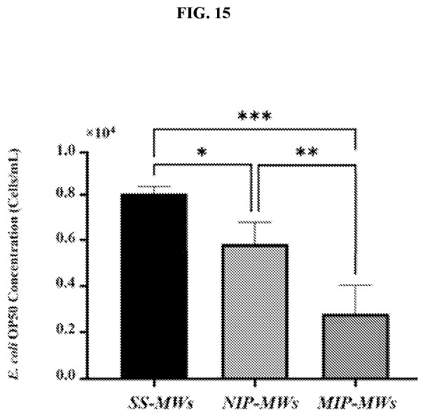

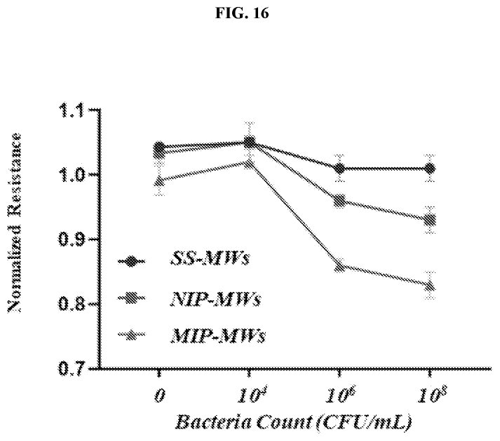

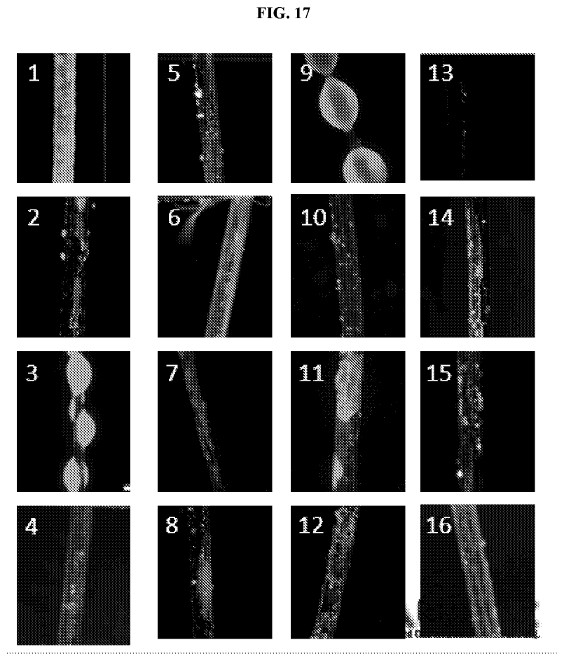

SEM images showed the successful and uniform coating of the MWs with the described preparation recipe. The coating is uniform, and no cavities are observed. As shown in FIG. 15, the off-chip assay results indicated that MIPs-MW successfully removed 76% of E. coli OP50 cells from bacterial suspensions with 104 CFU/ml initial cell count. Although rebinding assay proves the affinity of MIPs to the bacteria, transduction needs to occur to develop a sensing platform. FIG. 16 illustrates the results of the microfluidic biosensor platform developed. A significant difference (p-value<0.001) was observed in the resistance of the MIP-MWs device post bacteria application, indicating the capturing ability of the wires and the transduction of the device. The limit of detection (LOD) and the limit of quantification (LOQ) of the developed biosensor were calculated as 3.1×10⁵ CFU/mL and 4.3×10⁵ CFU/mL, respectively.

### Example 8: Capturing Sarcina Bacteria on MIP Wires

Surface treated SS microwires were prepared by subjecting surface treated SS wire to the polymerization conditions in Table 4, below and in the presence of Sarcina bacteria. The template Sarcina bacteria were removed to generate Sarcina MIP SS microwires.

TABLE 4
Recipe for MIP coated SS wires: Sample #1

| | |
|---|---|
| Monomer (MAA, μL) | 253.11 |
| Cross linker (EGDMA, μL) | 566.34 |
| Solvent (Acetonitrile, mL) | 3.2778 |
| Initiator (mg) | 38.4 |
| Time (h) | 7 |
| Temperature (° C.) | 60 |
| pH | 3.97 |
| Thickness (μm) | 131.87 |
| SD | 0.34 |
| Uniformity % | 99.73 |
| Stability | Yes |

The Sarcina MIP SS microwires were then mixed with a solution of Sarcina bacteria (1.5×10³) and the uptake ratio measured.

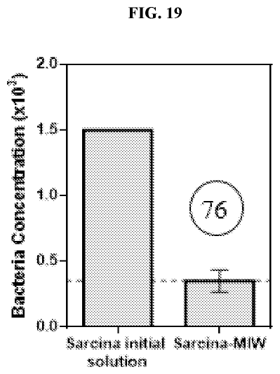

As shown in FIG. 19 Sarcina MIP SS microwires exhibited an uptake ratio of 76%.

What is claimed:

1. A molecularly imprinted polymer (MIP) coated article comprising:
(a) a substrate; and
(b) a MIP coating on the substrate;
wherein the MIP coating is prepared by polymerizing a reaction mixture comprising one or more binding monomers, one or more crosslinking monomers, at least one initiator, and at least one template having substantially the same size and shape as at least one target, on the surface of the substrate;
wherein the MIP coating has a plurality of imprinted cavities which selectively bind the at least one target and one or more properties selected from the group consisting of:
(i) an average thickness of about 0.01 μm to 100 μm
(ii) a uniformity of 90% or more;

2. A molecularly imprinted polymer (MIP) coated article comprising a substrate coated with a MIP polymer, prepared by a process comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as at least one target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
(i) an average thickness of 0.01 μm to 10 μm; and
(ii) a uniformity of 90% or more;
then removing the template from the polymer thereby forming a plurality of imprinted cavities in the MIP polymer which selectively bind at least one target.

3. The MIP coated article of claim 2, wherein one or more of steps (c) and (d) are carried out under the following conditions:
(i) said first polymerization conditions of step (c) is carried out at a temperature of about 20° C. to about 50° C.; and
(ii) said second polymerization conditions of step (d) is carried out at a temperature of about 50° C. to about 70° C.

4. The MIP coated article of claim 2 or 3, wherein one or more of steps (c) and (d) are carried out under the following conditions:
(i) said first polymerization conditions of step (c) is carried out at a temperature of about 40° C. to about 50° C.; and
(ii) said second polymerization conditions of step (d) is carried out at a temperature of about 50° C. to about 60° C.

5. The MIP coated article of claim 2 or 3, wherein one or more of steps (c) and (d) are carried out under the following conditions:
(i) said first polymerization conditions of step (c) is carried out at a temperature of about 40° C. to about 50° C. for about 4 to 6 hours; and
(ii) said second polymerization conditions of step (d) is carried out at a temperature of about 50° C. to about 60° C. for at least about 2 to 10 hours.

6. The MIP coated article of claim 2 or 3, wherein steps (c) and (d) are carried out under the following conditions:
(i) said first polymerization conditions of step (c) is carried out at a temperature of about 50° C.; for about 4 hours; and
(ii) said second polymerization conditions of step (d) is carried out at a temperature of about 60° C. for about 3 hours.

7. The MIP coated article of any one of claims 1-6, wherein steps (b), (c), and (d) are carried out in a porogen solvent.

8. The MIP coated article of any one of claim 1-7, wherein the substrate has a curved surface.

9. The MIP coated article of any one of claims 1-8, wherein the substrate is a microparticle or a microwire.

10. The MIP coated article of claim 9, wherein the substrate is surface functionalized with carboxyl groups.

11. The MIP coated article of any one of claims 1-10, wherein the target is a pathogen.

12. The MIP coated article of any one of claims 1-11, wherein the target is a bacteria or virus.

13. The MIP coated article of claim 12, wherein the bacteria is E. coli or Sarcina.

14. The MIP coated article of any one of claims 1-12, wherein the template is a biological target or a non-pathogenic strain of the target.

15. The MIP coated article of any one of claims 1-14, wherein the template is bacteriophage T4.

16. The MIP coated article of any one of claims 1-15, wherein the mixture comprises two or more binding monomers.

17. The MIP coated article of claim 16, wherein the mixture comprises four binding monomers.

18. The MIP coated article of any one of claims 1-17, wherein the binding monomers are each independently selected from the group consisting of a charged binding monomer, a hydrophilic binding monomer and a hydrophobic binding monomer.

19. The MIP coated article of claim 18, wherein the charged binding monomer comprises an anionic functional group.

20. The MIP coated article of claim 18, wherein the charged binding monomer comprises a cationic functional group.

21. The MIP coated article of claim 19, wherein the charged binding monomer is acrylic acid or an alkylacrylic acid.

22. The MIP coated article of claim 20, wherein the charged binding monomer is methacrylic acid.

23. The MIP coated article of claim 18, wherein the one or more hydrophilic binding monomers and one or more hydrophobic binding monomers are selected from the group consisting of acrylamide, methacrylamide, vinylpyridine, N-vinylpyrrolidone, N-alkylacrylamides, hydroxyethylmethacrylate, alkylmethacrylates, ethylmethacrylate and combinations thereof.

24. The MIP coated article of any one of claims 1-23, wherein the MIP coating is prepared by polymerizing on the surface of the substrate a mixture comprising:
(i) one or more monomers selected from the group consisting of acrylamide, methacrylic acid, methylmethacrylate, and N-vinylpyrrolidone, and combinations thereof,
(ii) one or more crosslinking monomers,
(iii) at least one initiator, and
(iv) at least one template having substantially the same size and shape as the target.

25. The MIP coated article of any one of claims 1-24, wherein the one or more crosslinking monomers are selected from the group consisting of ethylene glycol dimethacrylate (EGDMA), N,N′-methylenebisacrylamide, N,N′-(1,2-dihydroxyethylene)bisacrylamide, and combinations thereof.

26. The MIP coated article of any one of claims 1-25, wherein the initiator is a thermally activated.

27. The MIP coated article of any one of claims 1-25, wherein the initiator is a photoinitiator that is activated at a non-germicidal wavelength.

28. The MIP coated article of any one of claims 1-25, wherein the initiator is selected from the group consisting of azoisobutyronitrile (AIBN), diphenyl(2,4,6-trimethylbenzoyl)phosphine oxide (TPO), 1-phenyl 1,2-propanedione (PPD), camphorquinone (CQ), and phenylbis(2,4,6-trimethylbenzoyl)phosphine oxide, and combinations thereof.

29. The MIP coated article of any one of claims 1-28, wherein the reaction mixture comprises a detection enabling tag.

30. The MIP coated article of claim 29, wherein the detection enabling tag is a fluorescent dye.

31. MIP coated article of any one of claims 1-30, wherein the selectivity of the MIP is at least 70%.

32. MIP coated article of any one of claims 1-31, wherein the selectivity of the MIP is at least 90%.

33. The MIP coated article of any one of claims 1-32, wherein the average thickness of the MIP coating is about 0.5 μm to 10 μm.

34. The MIP coated article of any one of claims 1-33, wherein the average thickness of the MIP coating is about 2 μm to 4 μσm.

35. A sensor comprising the MIP coated article of any one of claims 1-34 and a transduction device, wherein when a target selectively binds to at least a portion of the imprinted cavities of the MIP coated article, the transduction device produced a signal.

36. A process for preparing a MIP coated article, comprising:
(a) providing a substrate functionalized to react with the MIP coating;
(b) contacting the substrate with a reaction mixture comprising: one or more binding monomers, one or more crosslinking monomers, at least one initiator, and at least one template having substantially the same size and shape as the target;
(c) reacting the reaction mixture under first polymerization conditions whereby the reaction mixture oligomerizes on the surface of the substrate;
(d) reacting the mixture under at least a second polymerization condition thereby forming a MIP polymer layer having one or more properties selected from the group consisting of:
(i) an average thickness of 0.01 μm to 10 μm; and
(ii) a uniformity of 90% or more;
then removing the at least one template from the MIP polymer layer thereby forming a plurality of imprinted cavities in the MIP polymer layer which selectively bind at least one target.

37. A method for detecting a pathogen, comprising contacting a sensor of claim 35, wherein the at least one target is a pathogen, with a fluid sample, wherein if the sample contains an amount of pathogen corresponding to at least the lower detection limit of the pathogen, the transduction device provides a signal indicating the presence of the pathogen, and if the sample contains an amount of pathogen below the lower detection limit of the pathogen, the transduction device does not provide a signal.
